(19) United States
(12) Patent Application Publication
(10) Pub. No.: US 2026/0026943 A1
(43) Pub. Date: Jan. 29, 2026

(54) EXPANDABLE IMPLANTS

(71) Applicant: NuVasive, INC., San Diego, CA (US)

(72) Inventors: Christopher Besaw, San Diego, CA (US); Thomas Sweeney, San Diego, CA (US); Scott Uyekawa, San Diego, CA (US); Garrett Mikels, San Diego, CA (US); Steven Lillig, San Diego, CA (US)

(21) Appl. No.: 19/345,157

(22) Filed: Sep. 30, 2025

Related U.S. Application Data
(63) Continuation of application No. 17/657,679, filed on Apr. 1, 2022, now Pat. No. 12,427,036.
(60) Provisional application No. 63/170,532, filed on Apr. 4, 2021.
(60) Provisional application No. 63/170,570, filed on Apr. 5, 2021.
(60) Provisional application No. 63/249,129, filed on Sep. 28, 2021.

(51) Int. Cl. A61F 2/44; A61F 2/30

(52) U.S. Cl. CPC A61F 2/447; A61F 2002/30405; A61F 2002/30484; A61F 2002/30538; A61F 2002/30556

(57) ABSTRACT

An expandable implant includes: a first endplate, a second endplate, a first translating member moveably coupled to a first actuator, and a second translating member moveably coupled to a second actuator. The first translating member may be configured to move independently from the second translating member, and the first translating member and the second translating member may be configured to change a spatial relationship between the first endplate and the second endplate. The change in spatial relationship may change a resulting height, length, width, angle of lordosis, or other dimension of the expandable implant.

### CROSS REFERENCE TO RELATED APPLICATIONS

The present patent application is a continuation application U.S. patent application Ser. No. 17/657,679, filed Apr. 1, 2022, which claims priority to U.S. Provisional Patent Application No. 63/170,532, filed Apr. 4, 2021; U.S. Provisional Patent Application No. 63/170,570, filed Apr. 5, 2021; and U.S. Provisional Patent Application No. 63/249,129, filed Sep. 28, 2021.

### BACKGROUND OF THE INVENTION

### Field of the Invention

The present disclosure relates generally to medical implants, and more particularly to expandable implants.

### Description of the Related Art

Back problems are one of the most common and debilitating occurrences in people of all ethnicities. In the United States alone, over 500,000 spine lumbar and cervical fusion procedures are performed each year. One of the causes of back pain and disability results from the rupture or degeneration of one or more intervertebral discs in the spine.

Surgical procedures are commonly performed to correct problems with displaced, damaged, or degenerated intervertebral discs due to trauma, disease or aging. Generally, spinal fusion procedures involve removing some or all of the diseased or damaged disc, and inserting one or more intervertebral implants into the resulting disc space. Anterior lumbar interbody fusion (ALIF) and lateral lumbar interbody fusion procedures are two of the techniques that spine surgeons use to access the portions of the spine to be repaired or replaced.

Replacement of injured or deteriorated spinal bone with artificial implants involves understanding and consideration of the mechanisms of the stresses inherent in the spine, as well as the biological properties of the body in response to the devices. Further, the size, configuration, and placement of an artificial implant involve precision positioning and handling by a skilled surgeon.

### SUMMARY OF THE INVENTION

This disclosure includes expandable implant devices and methods for using the same. The expandable implant device may be adjusted to form a particular height and/or a particular lordosis angle, as influenced by, inter alia, the needs or requirements of the patient, the target procedure of a surgeon, and may incorporate various features to accommodate spinal fusion.

In some embodiments, an expandable implant includes: a first endplate, a second endplate, a first translating member moveably coupled to a first actuator, and a second translating member moveably coupled to a second actuator. The first translating member may be configured to move independently from the second translating member, and the first translating member and the second translating member may be configured to change a spatial relationship between the first endplate and the second endplate. The spatial relationship may define a height, length, width, angle of lordosis, or any other dimension of the expandable implant.

In some embodiments, an expandable implant includes: a first endplate, a second endplate, a first translating member disposed between the first endplate and the second endplate and moveably coupled to a first drive screw, and a second translating member disposed between the first endplate and the second endplate and moveably coupled to a second drive screw. The first translating member is configured to move independently from the second translating member, and the first translating member and the second translating member are configured to change a spatial relationship between the first endplate and the second endplate.

In some embodiments, anti-migration features are provided to restrict undesired rotation of the lead screws. The anti-migration features provide added rigidity and increase an amount of load the expandable implant is configured to support.

In some embodiments, the first translating member and the second translating member may be moveably coupled at the distal end of the expandable implant by a dovetail coupling. Similarly, in some embodiments, the first translating member and the second translating member may be moveably coupled at the proximal end of the expandable implant by a dovetail coupling slideably coupling a first actuator housing to a second actuator housing.

In one embodiment of a method is provided for treating a spinal condition using an expandable implant, the method including the steps of: accessing an intervertebral disc space via a generally lateral approach, inserting an expandable implant into the intervertebral disc space, the expandable implant having a first translating member moveably coupled to a first actuator and a second translating member moveably coupled to a second actuator, with the first translating member and the second translating member configured to change a spatial relationship between the first endplate and the second endplate as they move, and actuating at least one of the first actuator and the second actuator to change the spatial relationship between the first endplate and the second endplate to change a dimension of the expandable implant.

### BRIEF DESCRIPTION OF THE DRAWINGS

These and other features will be further understood by those with skill in the art upon a review of the appended drawings, wherein:

FIG. 1 shows a side view of the expandable implant in accordance with the first embodiment, in which the expandable implant is collapsed in a first configuration;

FIG. 2 shows a side view of the expandable implant in accordance with the first embodiment, in which the expandable implant is expanded in a second configuration;

FIG. 3 shows a front perspective view of the expandable implant in accordance with the first embodiment, in which the expandable implant is expanded in a second configuration;

FIG. 4 shows a rear perspective view of the expandable implant in accordance with the first embodiment, in which the expandable implant is expanded in a second configuration;

FIG. 5 shows a top view of the expandable implant in accordance with the first embodiment;

FIG. 6 shows a cross-sectional top view of the expandable implant in accordance with the first embodiment;

FIG. 7 shows a top view of an expansion mechanism of the expandable implant in accordance with the first embodiment;

FIG. 8 shows a perspective view of a first translating member and a second translating member of an expandable implant in accordance with the first embodiment;

FIG. 9 shows a perspective view of an actuator housing of an expandable implant in accordance with the first embodiment;

FIG. 10 shows a rear view of the expandable implant in accordance with the first embodiment, which is collapsed in the first configuration;

FIG. 11 shows a rear view of the expandable implant in accordance with the first embodiment, which is expanded in the second configuration;

FIG. 12 shows a front view of the expandable implant in accordance with the first embodiment, which is collapsed in the first configuration;

FIG. 13 shows a front view of the expandable implant in accordance with the first embodiment, which is expanded in the second configuration;

FIG. 14 shows a rear view of the expandable implant in accordance with the first embodiment adjusted to an exemplary angle of lordosis;

FIG. 15 shows a front view of the expandable implant in accordance with the first embodiment adjusted to an exemplary angle of lordosis;

FIG. 16 shows a perspective view of the expandable implant in accordance with the first embodiment adjusted to an exemplary angle of lordosis;

FIG. 17 shows a rear perspective view of an expandable implant in accordance with a second embodiment, which is collapsed in a first configuration;

FIG. 18 shows a rear perspective view of an expandable implant in accordance with a second embodiment, which is expanded in a second configuration;

FIG. 19 shows a rear view of the expandable implant in accordance with the second embodiment, which is expanded in the second configuration;

FIG. 20 shows a rear view of the expandable implant in accordance with the second embodiment adjusted to the third configuration having an angle of lordosis;

FIG. 21 shows an actuator housing of an expandable implant in accordance with a third embodiment having an anti-rotation feature configured to restrict a rotation of the drive screws;

FIG. 22 shows a perspective view of an expandable implant in accordance with the third embodiment;

FIG. 23 shows an expandable implant in accordance with a fourth embodiment, the expandable implant having a sliding interface;

FIG. 24 shows the expandable implant in accordance with the fourth embodiment adjusted to an angle of lordosis;

FIG. 25 shows an expandable implant in accordance with a fourth embodiment, the actuator housing including a dove tail coupling configured to move independently;

FIG. 26 shows a top view of the expandable implant in accordance with the fourth embodiment;

FIG. 27 shows a top view of the expandable implant in accordance with the fourth embodiment in an adjusted configuration;

FIG. 28 shows a top view of an expandable implant in accordance with the fifth embodiment including a hinge joint;

FIG. 29 shows a front perspective view of the expandable implant in accordance with a fifth embodiment, the expandable implant being in a collapsed configuration;

FIG. 30 shows a side perspective view of the expandable implant in accordance with the fifth embodiment, the expandable implant being in an expanded configuration;

FIG. 31 shows an exploded view of the expandable implant in accordance with the fifth embodiment;

FIG. 32 shows a cross-sectional top view of the expandable implant in accordance with the fifth embodiment;

FIG. 33 shows a top-down view of the expandable implant in accordance with the fifth embodiment, with the top plate removed;

FIG. 34 shows an enlarged view of the box A of FIG. 33;

FIG. 35 shows a rear perspective view of the expandable implant in accordance with the fifth embodiment, the expandable implant having enhanced surface structure;

FIG. 36 shows a rear perspective side view of the expandable implant in accordance with a sixth embodiment, the expandable implant being plated;

FIG. 37 shows perspective side view of the expandable implant in accordance with a seventh embodiment, the expandable implant being plated;

FIG. 38 shows an enlarged perspective view of the fixation aperture in accordance with an embodiment of the disclosure;

FIG. 39 shows a cross-sectional view of the fixation aperture of FIG. 38;

FIG. 40 shows a perspective view of a fixation element in accordance with an embodiment of the disclosure, including an enlarged view of the head of the fixation element;

FIG. 41 shows a perspective top view of a fixation aperture according to another embodiment;

FIG. 42 shows a perspective top view of a fixation aperture according to another embodiment;

FIG. 43 shows a side view of a fixation element according to another embodiment;

FIG. 44 shows a cross-sectional view of the head of the fixation element according to the embodiment of FIG. 43;

FIG. 45 shows a top-down view of the fixation element according the embodiment of FIG. 43;

FIG. 46 shows a rear view of an expandable implant in accordance with an eighth embodiment;

FIG. 47 shows a perspective side view of a modular plate design of an expandable implant in accordance with a ninth embodiment;

FIG. 48 shows a perspective side view of the modular plate coupled with the housing in accordance with the ninth embodiment;

FIG. 49 shows an exploded view of the modular plate with the housing in accordance with the ninth embodiment;

FIG. 50 shows a cross-sectional top view of the modular plate coupled with the housing in accordance with the ninth embodiment; and

FIG. 51 shows a cross-sectional side view of the modular plate coupled with the housing in accordance with the ninth embodiment.

### DETAILED DESCRIPTION

For purposes of explanation and not limitation, details and descriptions of certain preferred embodiments are hereinafter provided such that one having ordinary skill in the art may be enabled to make and use the invention. These details and descriptions are representative only of certain preferred embodiments. However, a myriad of other embodiments which will not be expressly described will be readily understood by those having skill in the art upon a thorough review hereof. Accordingly, any reviewer of the instant disclosure should interpret the scope of the invention by the claims, and such scope shall not be limited by the embodiments described and illustrated herein.

This disclosure includes expandable implant devices and methods for using the same. The expandable implant device may be adjusted to form a particular height and a particular lordosis angle, as influenced by: the needs or requirements of the patient, the target procedure of a surgeon, etc., and the expandable implant may further incorporate various features to accommodate and/or promote spinal fusion.

In a general embodiment, an expandable implant includes: a first endplate, a second endplate, a first translating member, and a second translating member, wherein the first translating member is configured to move independently of the second translating member, and wherein movement of at least one of the first translating member and the second translating member is configured to change a dimension of the expandable implant.

In some embodiments, an expandable implant includes: a first endplate, a second endplate, a first actuator having a first translating member moveably coupled to the first actuator, and a second actuator having a second translating member moveably coupled to the second actuator with the first translating member configured to move independently from the second translating member, and the first translating member and the second translating member configured to change a spatial relationship between the first endplate and the second endplate. The spatial relationship may include a dimension of the expandable implant, and may include at least one of height, width, length, lordosis, and any other dimension of the expandable implant.

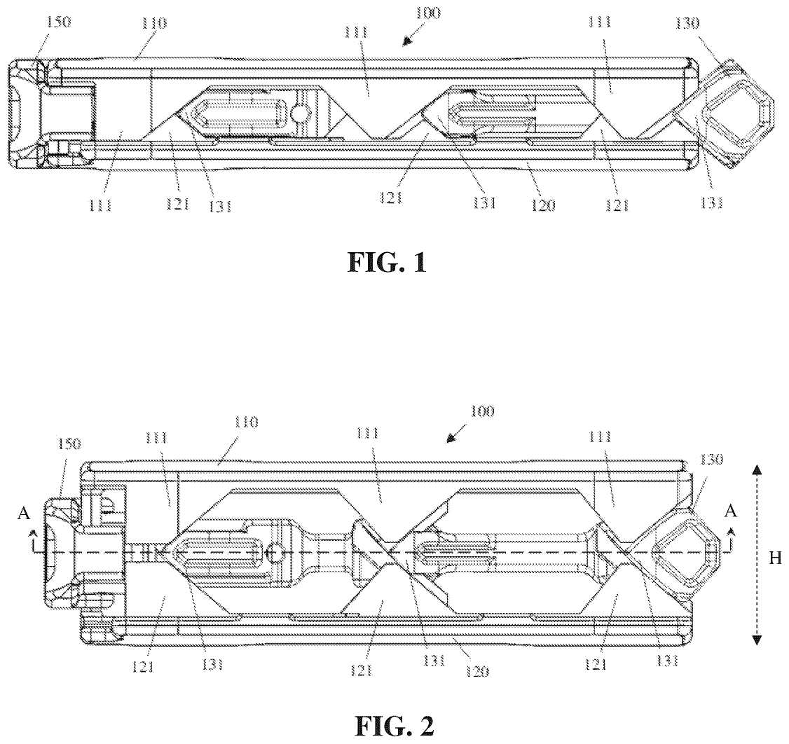

Now turning to the drawings, FIG. 1-13 show various views of an expandable implant 100 in accordance with a first embodiment.

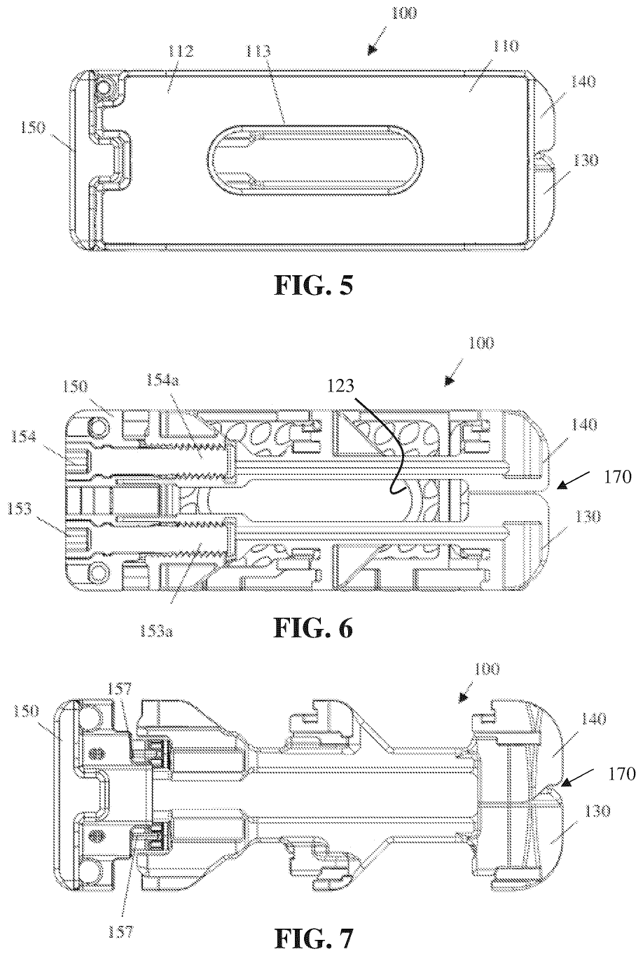

FIG. 1 shows an expandable implant 100 collapsed in a first configuration. Expandable implant 100 is shown including a first endplate 110, a second endplate 120, a first translating member 130 (FIGS. 5-8) and a second actuator having a second translating member 140 (FIGS. 5-8). First translating member 130 may be configured to move independently from second translating member 140, and first translating member 130 and second translating member 140 may be configured to change a spatial relationship between first endplate 110 and second endplate 120.

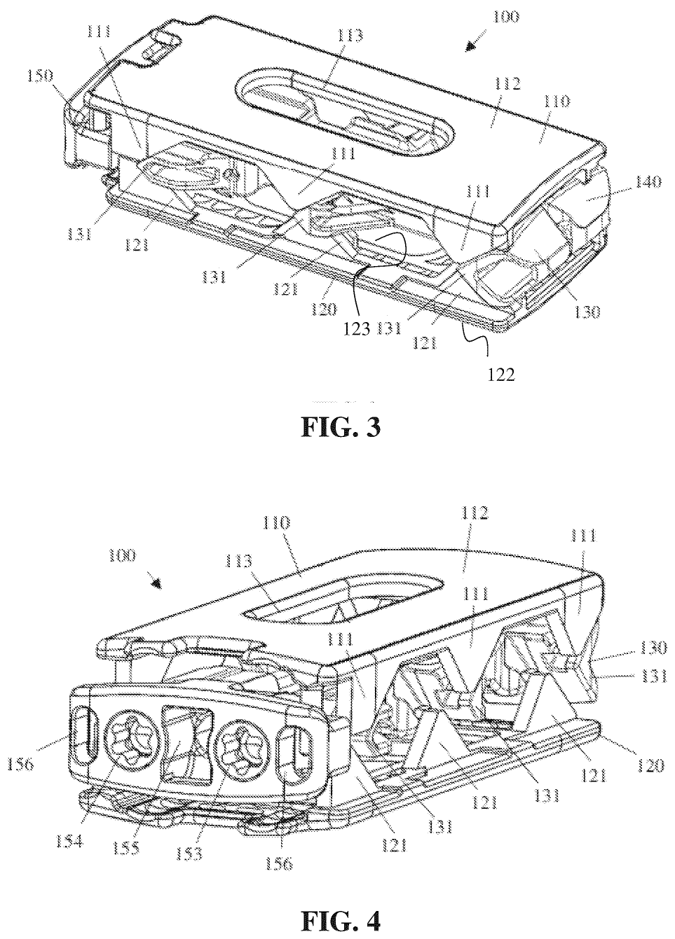

The expandable implant 100 may be inserted through an access incision in a collapsed configuration to reduce an insertion profile of expandable implant 100. The first endplate 110 and the second endplate 120 further include a fusion aperture 113 (FIGS. 3-5), 123 (FIG. 3) and a bone contact surface 112 (FIGS. 3-5), 122 (FIG. 3). After placing the expandable implant 100 within a prepared intervertebral disc space of a patient, and adjusting the expandable implant 100 to a desired height and angle of lordosis, the bone contact surfaces 112, 122 of the first endplate 110 and the second endplate 120, respectively, are configured to contact an inferior vertebral body and a superior vertebral body respectively. As such, the bone contact surfaces 112, 122 may further include anti-migration features configured to prevent slippage of the expandable implant 100 within the intervertebral disc space of the patient. The bone contact surfaces 112, 122 may further include a porous bone contact surface to promote fusion.

FIG. 2 shows a side view of the expandable implant 100 expanded in a second configuration, to a height H. Here, and as described above, the threaded portion of the first drive screw 153 (FIGS. 6 and 9) and the threaded portion of the second drive screw 154 (FIGS. 6 and 9) have been driven into the threaded cavity of the first translating member 130 and the threaded cavity of the second translating member 140 respectively. Communication of the threaded portion 153a (FIGS. 6 and 9) of the first drive screw 153 with the threaded cavity of the first translating member 130 moves the first threaded portion towards the actuator housing 150 (FIGS. 6-7 and 9). Similarly, communication of the threaded portion 154a of the second drive screw 154 with the threaded cavity of the second translating member 140 moves the second threaded portion towards the actuator housing 150.

FIG. 3 shows a front perspective view of the expandable implant 100 in accordance with the first embodiment. Here, the expandable implant 100 is shown in the second, expanded configuration. Rotation of the first drive screw 153 may drive the threaded portion 153a of the first drive screw 153 into the threaded cavity of the first translating member 130. The position of the first drive screw 153 relative to the actuator housing 150 may be fixed, with the drive screw 153 captured but rotatably disposed about the actuator housing 150. Driving the threaded portion 153a of the first drive screw 153 into the translating member 130 effectively moves the translating member 130 along the length of the first drive screw 153 toward the actuator housing 150, changing a dimension of the expandable implant 100.

FIG. 4 shows a rear perspective view of the expandable implant 100 in accordance with the first embodiment, having a first actuator including a first drive screw 153 (FIGS. 6 and 9) and a second actuator including a second drive screw 154 (FIGS. 6 and 9). The actuator housing 150 includes one or more apertures configured to receive at least a portion of the first drive screw 153 and the second drive screw 154 therethrough. The actuator housing 150 is moveably connected to the first endplate 110 and the second endplate 120, allowing movement of the first endplate 110 and the second endplate 110 upon an actuation of the first drive screw 153 and the second drive screw 154.

The actuator housing 150 further includes inserter cavities 156 and a graft aperture 155. The inserter cavities 156 are configured to removably secure the expandable implant 100 to a tip of the inserter. After placing the expandable implant 100 within a prepared intervertebral disc space of a patient, and adjusting the expandable implant 100 to a desired height and angle of lordosis, bone graft or bone graft substitute material may be loaded into an inner volume of the expandable implant 100. In some embodiments the bone graft or bone graft substitute material is delivered to the expandable implant 100 by the inserter and loaded into the inner volume of the expandable implant 100 through the graft aperture 155.

The bone fusion apertures 113, 123 of the first endplate 110 and the second endplate 120 are configured to allow the bone graft or bone graft substitute material packed within the inner volume of the expandable implant to communicate with the vertebral bodies of the patient to assist in the bone fusion process.

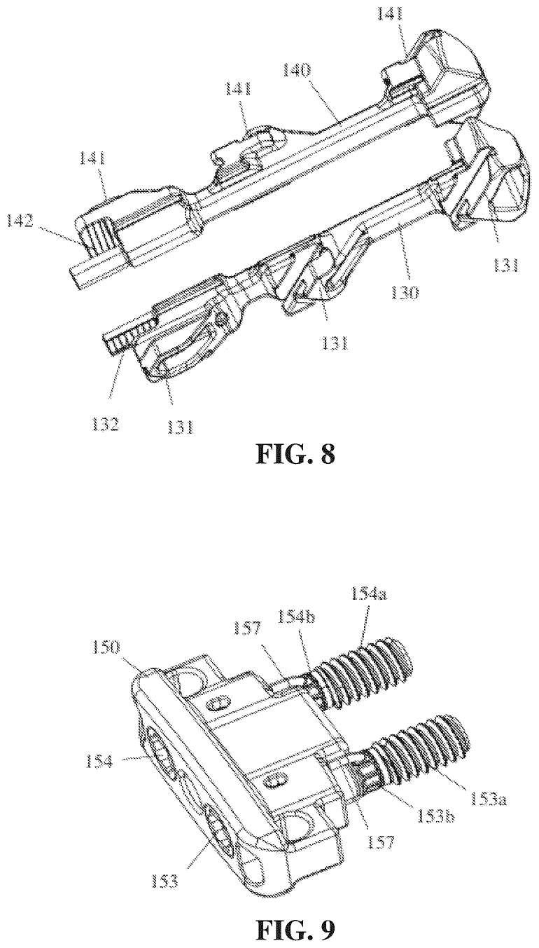

The first drive screw 153 and the second drive screw 154 each include a threaded portion 153a, 154a configured to nest within a threaded cavity 132, 142 (FIG. 8) of the first translating member 130 and the second translating member 140 respectively. For example, the threaded portion 153a of the first drive screw 153 is configured to communicate with the threaded cavity 132 of the first translating member 130, and the threaded portion 154a of the second drive screw 154 is configured to communicate with the threaded cavity 142 of the second translating member 140.

At least a portion of the first translating member 130 and the second translating member 140 may be disposed between the first endplate 110 and the second endplate 120. Each of the first translating member 130 and the second translating member 140 may include one or more wedges 131, 141 (FIG. 8) configured to communicate with at least one wedge 111, 121 (FIG. 1-4) of the first endplate 110 and the second endplate 120, upon a movement of the translating member 130, 140. In some embodiments, one or more of the first endplate 110 and the second endplate 120 may include at least one wedge 111, 121 configured to communicate with the one or more wedges 131, 141 of at least one translating member 130, 140.

The first translating member 130 and the second translating member 140 are slideably coupled at the distal end of the expandable implant 100, allowing the first translating member 130 to move independently from the second translating member 140. When the first translating member 130 is simultaneously moved in substantially the same direction and substantially the same distance as the second translating member 140, the one or more wedge 111, 121 of the first endplate 110 and the second endplate 120 will slide up the one or more wedge 131, 141 of the first translating member 130 and the second translating member 140, changing the height of the expandable implant 100. Simultaneous rotation of the first drive screw 153 and the second drive screw 154 is not required to selectively change the height, but the magnitude of distance traveled by the first translating member 130 and the second translating member 140 relative to the drive screws 153, 154 should be substantially equal to minimize an amount of angular actuation of the first endplate 110 relative to the second endplate 120.

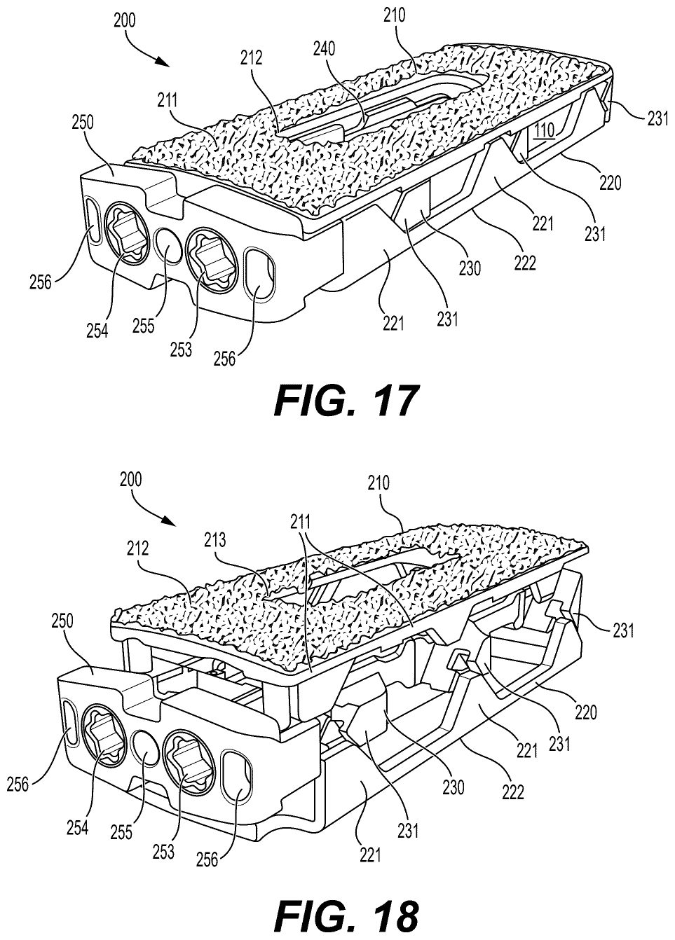

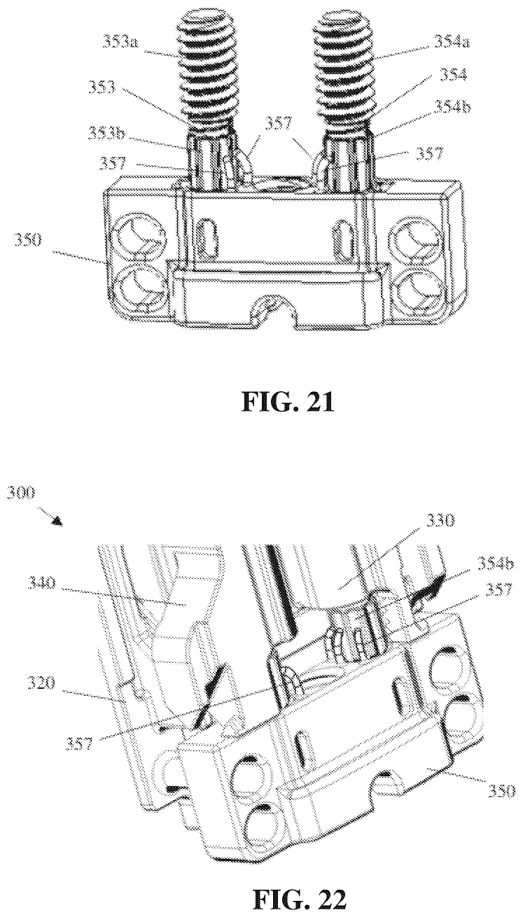

Angular actuation of expandable implant 100 may be achieved when one of first translating member 130 and second translating member 140 are moved and when first translating member 130 and second translating member 140 are moved in opposite directions, changing an orientation of first endplate 110 relative to second endplate 120. The change in orientation corresponds to a change in an angle of lordosis of expandable implant 100 (see FIGS. 17 and 21).

As one with skill in the art may appreciate, a patient's condition or other spinal deformity may require an implant of height H. However, the larger the implant, the larger the incision required to place the implant in the intervertebral space of the patient during surgery. Providing an expandable implant 100 allows a surgeon to insert the expandable implant 100 in the first, collapsed configuration, and then expand the expandable implant to the height H with the implant situated in the intervertebral space of the patient, thereby only requiring a minimal incision.

In some embodiments, at least one of first translating member 130 and second translating member 140 may include at least one wedge 131, 141. Each wedge 131, 141 may be configured to communicate with at least one of a wedge 111 of first endplate 110 and at least one wedge 121 of second endplate 120. Upon a movement of first translating member 130 toward actuator housing 150, the at least one wedge 131 of first translating member 130 is configured to communicate with at least one of first endplate 110 and second endplate 120. Similarly, upon of movement of second translating member 140 toward actuator housing 150, the at least one wedge 141 of second translating member 140 is configured to communicate with at least one of first endplate 110 and second endplate 120.

In this embodiment, substantially equal and simultaneous movement of the first translating member 130 and the second translating member 140 in the same direction will change the height and slightly vary the length of the expandable implant 100. The first translating member 130 and the second translating member 140 of this embodiment each include three wedges 131, 141 configured to communicate with opposing wedges 111, 121 of the first endplate 110 and the second endplate 120 upon a movement of the first translating member 130 and the second translating member 140 toward the actuator housing 150.

As one with skill in the art may appreciate, one or more of simultaneous movement of the first translating member 130 and the second translating member 140 in opposite directions, and movement of only one of the first translating member 130 and the second translating member 140, may change the height of each respective end of the expandable implant 100, thereby effectively changing the angle of the first endplate 110 relative to the second endplate 120. In some embodiments, the adjusted angle between the first endplate 110 and the second endplate 120 may include an angle of lordosis.

FIG. 5 shows a top view of the expandable implant 100 in accordance with the first embodiment. The first endplate 110 and the second endplate 120 shown including a fusion aperture 113, 123 (FIG. 6). The fusion aperture 113 is configured to assist in the bone fusion process, allowing bone graft or bone graft material deposited within an inner volume of the expandable implant, to communicate with the vertebral bodies of the patient. Offsetting the first translating member 130 and the second translating member 140 such that they extend along opposite ends, e.g., first and second ends of the expandable implant creates a cavity within an inner volume of the expandable implant 100. That cavity may be filled with bone graft or bone graft substitute material to aid in the fusion process.

FIG. 6 shows a cross-sectional top view of the expandable implant 100 taken across the plane A (see FIG. 2). Here, a threaded portion 153b of the first drive screw 153 and a threaded portion 154b of the second drive screw 154 are shown, nested within a threaded cavity of the first translating member 130 and a threaded cavity of the second translating member 140 respectively. In this embodiment, the first drive screw 153 and the second drive screw 154 are rotatably captured within the actuator housing 150. This arrangement allows rotational movement of the first drive screw 153 and the second drive screw 154 but prevents translational movement of the first drive screw 153 and the second drive screw 154.

FIG. 7 shows a top view of an expansion mechanism of the expandable implant 100 in accordance with the first embodiment. The expansion mechanism includes the actuator housing 150, the first drive screw 153, the first translating member 130, the second drive screw 154, and the second translating member 140.

As will be described in more detail below, this embodiment also includes an anti-rotation mechanism configured to restrict undesired rotation of the drive screws. In this embodiment the anti-rotation mechanism includes a plurality of flexible members 157, at least a portion of the plurality of flexible members 157 being configured to nest within a plurality of divots of the first drive screw 153 and the second drive screw 154.

FIG. 8 shows a perspective view of a first translating member 130 and a second translating member 140 of an expandable implant 100. First translating member 130 and second translating member 140 include a threaded aperture 132, 142 configured to communicate with threaded portion 153a, 154a of a drive screw 153, 154. Translating members 130, 140 each further include a plurality of wedges 131, 141 configured to communicate with wedges 111, 121 of first endplate 110 and second endplate 120 and configured to move first endplate 110 relative to second endplate 120.

FIG. 9 shows a perspective view of an actuator housing 150 of an expandable implant 100 having a first drive screw 153 and a second drive screw 154 nested therein. The actuator housing 150 shown includes a plurality of flexible members 157 configured to restrict undesired movement of the first drive screw 153 and the second drive screw 154.

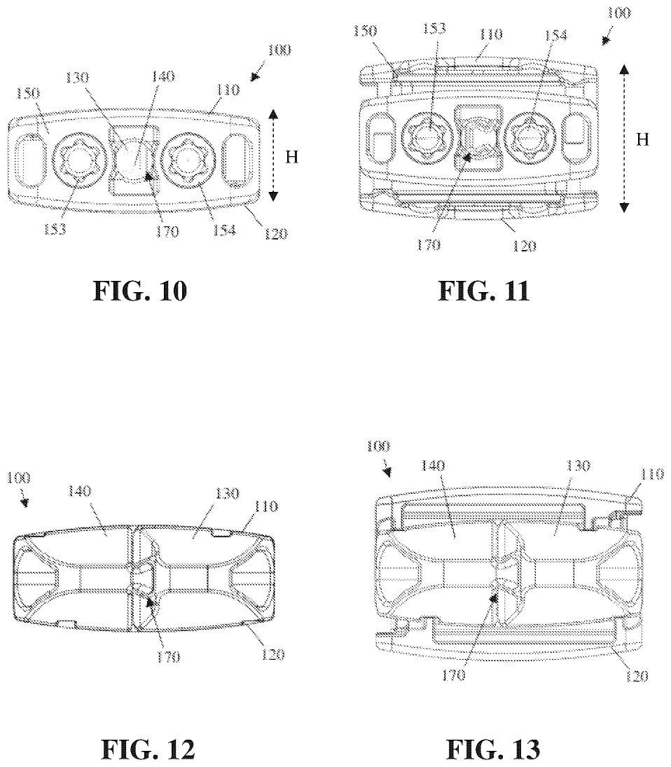

FIGS. 10-15 show two perspective views, front and back, of the expandable implant 100 as it is adjusted from the first, collapsed configuration to the second, expanded configuration. FIG. 10 shows a rear view of the expandable implant 100 collapsed in the first configuration. Note the relative locations of the first endplate 110 and the second endplate 120. FIG. 11 shows a rear view of the expandable implant 100 expanded in the second configuration. Note the movement of the first endplate 110 relative to the second endplate 120, and the change of height of the expandable implant 100.

As seen from another perspective, FIG. 12 shows a front view of the expandable implant 100 collapsed in the first configuration and FIG. 13 shows a front view of an expandable implant 100 expanded in the second configuration.

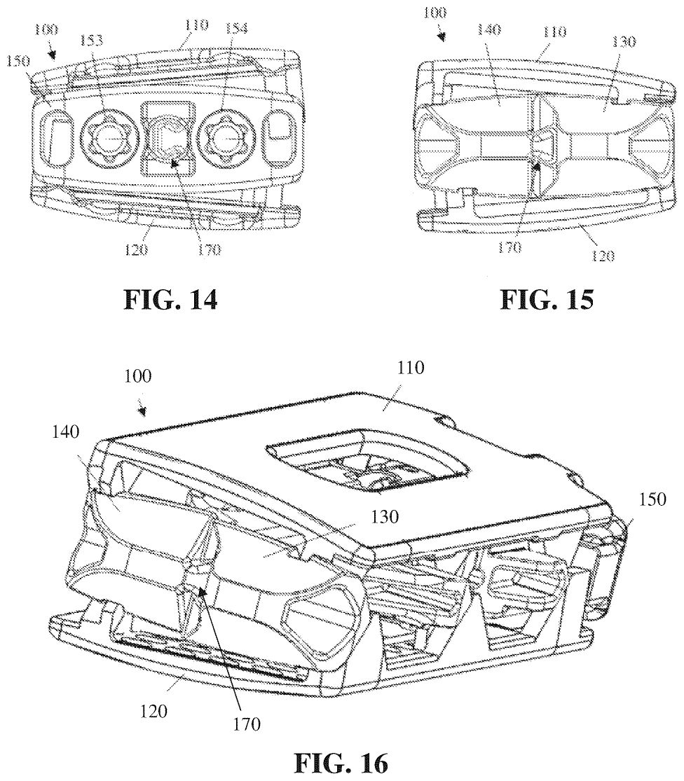

To illustrate angular adjustment of the expandable implant 100, FIG. 14 shows a rear view of the expandable implant 100 in accordance with the first embodiment, adjusted to an exemplary angle of lordosis. FIG. 15 shows a front view of expandable implant 100 in accordance with the first embodiment, adjusted to an exemplary angle of lordosis. By comparing this configuration with the first and second configurations described above, we see that first endplate 110 sits at an angle relative to second endplate 120, corresponding to an exemplary angle of lordosis.

FIG. 16 shows a perspective view of the expandable implant 100 in accordance with the first embodiment adjusted to an exemplary angle of lordosis. Depending on the needs of the patient, the individual components of the expandable implant may be sized to provide the required amount of lordosis.

The expandable implant 100 includes a slideable interface 170 (FIGS. 6-7 and 10-16) disposed at the distal end of the expandable implant 100. The distal end of the expandable implant 100, and particularly the distal ends of the first translating member 130 and the second translating member 140, are configured with a tapered edge having a slidable interface 170 moveably coupling the first translating member 130 to the second translating member 140. The tapered edge is configured to aid in insertion of the expandable implant into an intervertebral disc space of the patient, and the slideable interface 170 is configured to increase the stability and loadbearing characteristics of the expandable implant 100.

In this embodiment, the slidable interface 170 includes a dovetail coupling, the dovetail coupling including an extension of the first translating member 130 nested within a channel of the second translating member. As one with skill in the art may appreciate, in this embodiment the extension is a portion of the first translating member 130 and the channel is disposed on the second translating member 140. In some embodiments, the extension is a portion of the second translating member 140 and is in communication with a channel of the first translating member 130. Similarly, in this embodiment the slidable interface 170 is a dovetail coupling, having a substantially triangular geometry, and as one with skill in the art may appreciate other geometries may be provided.

FIGS. 17-20 show a second embodiment of an expandable implant 200 including: a first endplate 210, a second endplate 220, a first translating member 230 disposed between first endplate 210 and second endplate 220 and moveably coupled to a first drive screw 253, and a second translating member 240 disposed between the first endplate 210 and second endplate 220 and moveably coupled to a second drive screw 254. First translating member 230 is configured to move independently from second translating member 240, and first translating member 230 and second translating member 240 are configured to change a spatial relationship between first endplate 210 and second endplate 220. First and second translating members 230, 240 are mirror images of each other, and therefore, have corresponding features.

In this embodiment, the first endplate 210 and the second endplate 220 include a porous bone contact surface 211, 221. The first endplate 210, the second endplate 220, and any other component of the expandable implant 200 may be fabricated using additive manufacturing techniques. The porous bone contact surface 211, 221 may be printed using an additive manufacturing technique. In some embodiments, the first endplate 210, the second endplate 220, and any other component of the expandable implant 200 may comprise polyether ketone (PEEK). As such, the porous bone contact 211, 221 surface may be fabricated using any known process for manufacturing porous PEEK implants. In some embodiments, the expandable implant 200 may be manufactured from titanium, and any other material commonly used in fabrication of medical implants.

FIG. 17 shows the expandable implant 200 in a first collapsed configuration. The expandable implant 200 includes: a first endplate 210, a second endplate 220, a first translating member 230, a second translating member 240, and an actuator housing 250. The actuator housing 250 includes a first drive screw 253 with a first threaded portion in communication with a threaded cavity of the first translating member 230, and a second drive screw 254 in communication with a first threaded cavity of the second translating member 240.

Upon a rotation of at least one of the first drive screw 253 and the second drive screw 254, communication of the threaded portion (not shown in FIG. 17, but similar to 153a, 154a (FIG. 6) relative to implant 100) with the first threaded cavity 232, 242 of the first translating member 230 and the second translating member 240, thereby translating the first translating member 230 and/or the second translating member 240.

Each of the first translating member 230 and the second translating member 240 may include at least one wedge 231, 241, with each wedge 231, 241 configured to communicate with a wedge 211, 221 of the first endplate 210 and the second endplate 220. Upon a translation of one or more of the first translating member 230 and the second translating member 240, the one or more wedges 231, 241 of the translating members 230, 240 are configured to communicate with one or more wedges 211, 221 of the endplates 210, 220 and thereby change a dimension of the expandable implant 200. While not shown in FIGS. 17-18, it is to be understood that second translating member 240 includes wedges 241 since translating member 240 is a mirror image of translating member 230.

In this embodiment, the wedges 211, 221 of the endplates 210, 220 include a keyed portion configured to travel in a track of the wedges 231, 241 of the translating members 230, 240. The keyed portion traveling in the track is configured to provide additional stability and load support characteristics to the expandable implant.

FIG. 18 shows a rear perspective view of the expandable implant 200 in its second, expanded configuration. When the first drive screw 253 and the second drive screw 254 are rotated such the first translating member 230 and the second translating member 240 move a substantially equal amount, the wedges 231, 241 of the first translating member 230 and the second translating member 240 communicate with the wedges 211, 221 of the first endplate 210 and the second endplate 220, to displace the first endplate 210 and second endplate 220 relative to the actuator housing 250. In this embodiment, this movement will expand the expandable implant 200, changing the height of the expandable implant 200.

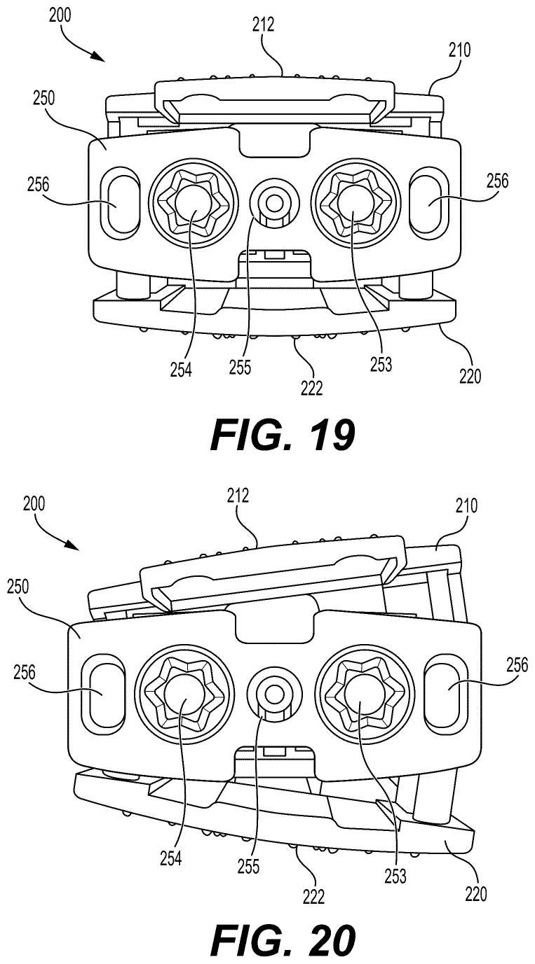

Now, in some embodiments the expandable implant 200 is also configured to be adjusted for an angle of lordosis. For reference, FIG. 19 shows a rear view of the expandable implant 200 in the same second, expanded configuration of FIG. 18. As one with skill in the art may appreciate, by either rotating one of the first drive screw 253 and the second drive screw 254, and by rotating both the first drive screw 253 and the second drive screw 254 in opposite directions, will change an angle between the first endplate 210 and the second endplate 220.

FIG. 20 shows a rear view of the expandable implant 200 adjusted to a third configuration having a chosen angle of lordosis. As one with skill in the art may appreciate, choosing the dimensions of the wedges 211, 221 of the endplates 210, 220, and the wedges 231, 241 of the translating members 230, 240 will determine a range of angles of lordosis the expandable implant 200 is configured to provide.

As one with skill in the art may appreciate, the first embodiment 100, the second embodiment 200, and all embodiments shown and described herein may be adjusted in both height and angle of lordosis. Any number of configurations of height and lordosis may be provided by the expandable implant, but may not be shown explicitly herein. In this sense, the full range of possible adjustments should be viewed as being encompassed by this disclosure, and the disclosure is not to be limited by the configurations explicitly shown herein.

Some embodiments may include anti-rotation features to restrict undesired rotation of the lead screws. The anti-rotation features provide added rigidity and increase an amount of load the expandable implant is capable of supporting. For example, FIG. 21 and FIG. 22 show an actuator housing 350 of an expandable implant 300 in accordance with a third embodiment, the actuator housing 350 including an anti-rotation feature 357 configured to restrict an undesired rotation of the first drive screw 353 and the second drive screw 354. In this embodiment, the anti-rotation features 357 include a plurality of flexible members 357 extending from the actuator housing 350. The first drive screw 353 and the second drive screw 354 of this embodiment include a plurality of divots 353b, 354b, each configured to receive at least a portion of each of the plurality of flexible members 357 therein with the anti-rotation feature configured to ratchet and prevent undesired rotation of the drive screws 353, 354.

As one with skill in the art may appreciate, communication of at least a portion of each of the plurality of flexible members 357 nesting within the plurality of divots 353b, 354b increases the force requirement needed to rotate the drive screws 353, 354. However, the ratchet mechanism of the plurality of flexible members 357 communicating with the divots 353b, 354b of the drive screws 353, 354, acts to prevent undesired rotation of the drive screws 353, 354, and helps prevent collapse of an expandable implant 300.

In some embodiments, the actuator housing 350 may be fabricated using additive manufacturing techniques, and the plurality of flexible members 357 may be fabricated such that the actuator housing 350 and the plurality of flexible members 357 form a monolithic piece. In some embodiments, this single monolithic piece may be 3D printed directly around the drive screws, to reduce the number of components and aid in the assembly process. In some embodiments, the drive screws are inserted through respective cavities and seated into place with a friction fit.

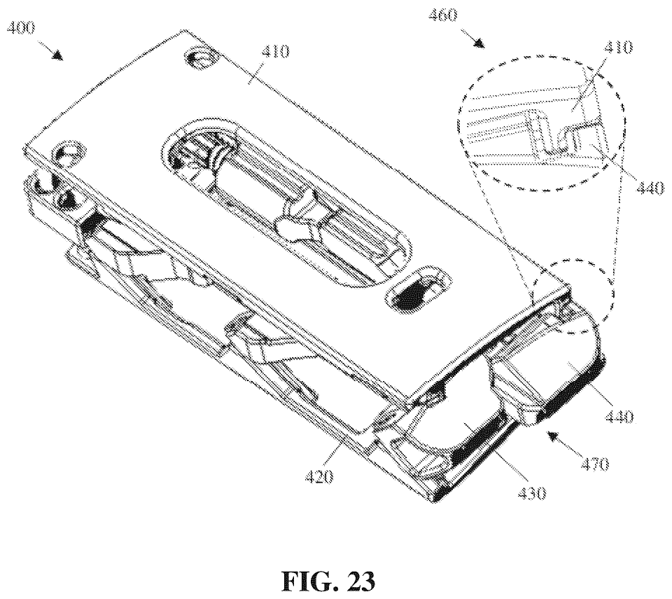

FIG. 23 shows an expandable implant 400 in accordance with a fourth embodiment, adjusted to a chosen height and angle of lordosis. The expandable implant 400 includes a sliding interface 460 formed at the interface of the ends of the endplates 410, 420 and each of the translating members 430, 440. An enhanced view of the sliding interface 460 of the first endplate 410 and the second translating member 440. This sliding interface 460 between the endplates 410, 420 and the translating members 430, 440 helps the expandable implant 400 achieve an increased amount of lordosis.

In this embodiment, sliding interfaces 460 are formed by providing at least one groove along the length of the translating members 430, 440 and a plurality of notches extending along at least a portion of the length of endplates 410, 420. As the expandable implant is adjusted to particular angle of lordosis, the pivot point of the first endplate 410 and the second endplate 420 will change. As the pivot point moves toward an end of the endplate 410, 420, the translating member 430, 440 may abut and effectively restrict movement of the endplate 410, 420. However, by providing a sliding interface 460, the endplate is provided an increased amount of travel.

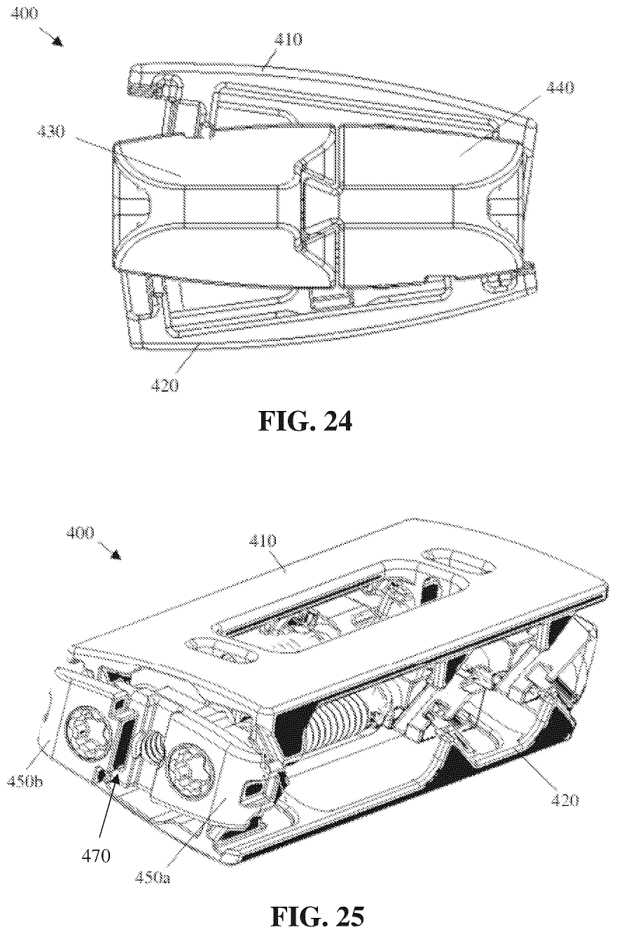

In some embodiments, the first translating member 430 and the second translating member 440 may be moveably coupled at the distal end of the expandable implant by slideable interface 470. The slideable interface 470 may be configured to allow the first translating member 430 and the second translating member 440 to move independently. FIG. 24 shows the distal end of the expandable implant 400, the slideable interface 470 (FIG. 25) having a dovetail coupling that slideably connects the first translating member 430 and the second translating member 440. The dovetail coupling is configured to allow the first translating member 430 and the second translating member 440 to move independently. Similarly, in some embodiments the first translating member 430 and the second translating member 440 may be moveably coupled at the proximal end of the expandable implant by a slideable interface configured to allow the first translating member 430 and the second translating member 440 to move independently.

In some embodiments, a slideable interface 470 at a proximal end of the expandable implant 400 maybe formed between a first actuator housing 450a and a second actuator housing 450b (FIGS. 25-27). FIG. 25 shows a proximal end of the expandable implant 400 having a slideable interface 470 configured to provide a moveable coupling between a first actuator housing 450a and a second actuator housing 450b. The slideable interface 470 includes a dovetail coupling configured to allow the first actuator housing 450a and the second first actuator housing 450b to move independently.

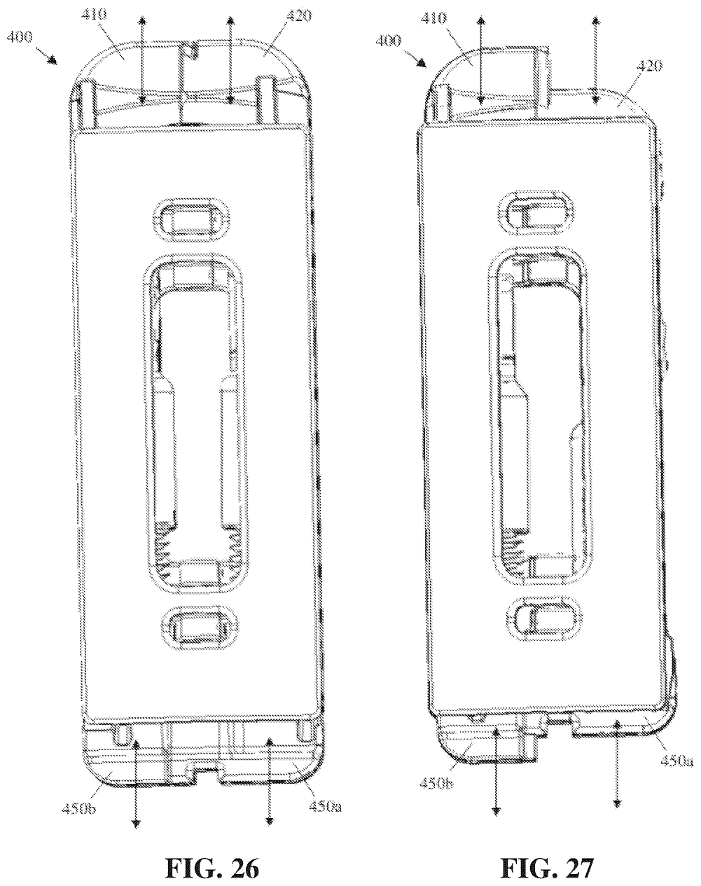

Providing a slideable coupling at both the proximal and distal ends of the expandable implant provides additional stability and load bearing characteristics to the expandable implant. Additionally, providing a slideable coupling at both the proximal and distal ends of the expandable implant may allow length expansion and length contraction of the expandable implant at either end of the expandable implant. For example, FIGS. 26-27 show top views of the expandable implant 400 in accordance with the fourth embodiment. In FIG. 26, the expandable implant 400 is arranged in a first collapsed configuration. In FIG. 27, the first drive screw has been rotated, to adjust an angle of lordosis of the expandable implant 400. As such, the first actuator housing 450a has moved toward the first translating member 430, pivoting the first endplate 410 and the second endplate 420.

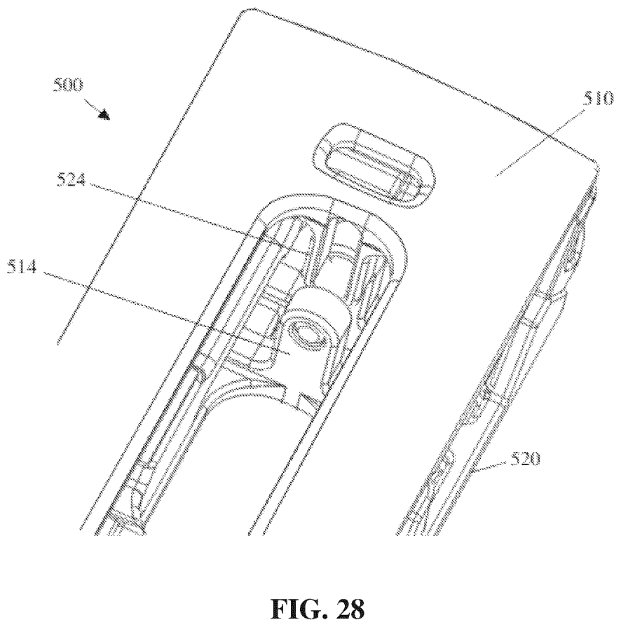

With reference to FIG. 28, in some embodiments, one or more hinge joints may be provided. The hinge joint may be configured to provide additional stability to the expandable implant. The hinge joint may moveably couple the first endplate 510 to the second endplate 520. In some embodiments, the first endplate 510 and the second endplate 520 may be coupled at the posterior end or the anterior end. In some embodiments, the first endplate 510 and the second endplate 520 may be moveably coupled by linkages. For example, FIG. 28 shows an expandable implant 500 in accordance with a fifth embodiment, the first endplate 510 having a linkage 514 moveably coupled to a linkage 524 of the second endplate 520. The linkage 514 of the first endplate 510 is moveably coupled to the linkage 524 of the second endplate 520 by a coupling pin. These linkages 514, 524 moveably couple the first endplate 510 to the second endplate 520 and improve stability and load bearing characteristics of the expandable implant 500.

A method is further provided for treating a spinal condition using an expandable implant. The method may include accessing an intervertebral disc space via a generally lateral approach, and inserting an expandable implant into the intervertebral disc space, the expandable implant having a first translating member moveably coupled to a first actuator and a second translating member moveably coupled to a second actuator. The first translating member and the second translating member may be configured to change a spatial relationship between the first endplate and the second endplate as they move. The method may further include actuating at least one of the first actuator and the second actuator to change the spatial relationship between the first endplate and the second endplate to change a dimension of the expandable implant.

In some embodiments, the method may include the step of adjusting the expandable implant in situ within the intervertebral disc space of the patient.

In some embodiments, the method may include the step of packing the expandable implant with bone graft or bone graft substitute material. This step may occur prior to inserting the implant into the disc space or after placing the implant in the disc space by using a specialized inserter to provide the bone graft or bone graft substitute material to the expandable implant in situ.

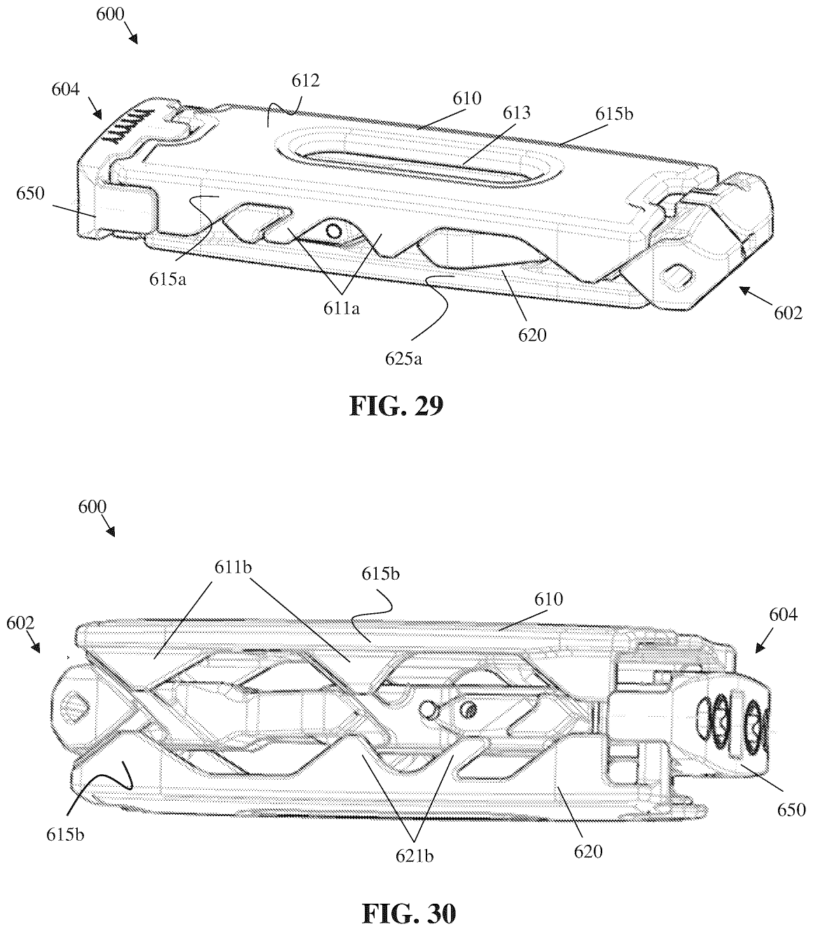

In some embodiments, the method may include the step of adjusting the expandable implant to a desired height, and may additionally or alternatively include the step of adjusting the expandable implant to a desired angle of lordosis. FIG. 29 illustrates an expandable fusion intervertebral implant device 600 according to another embodiment. As shown in FIG. 29, the expandable fusion intervertebral device 600 is in a collapsed, insertion configuration. FIG. 30 illustrates the expandable fusion intervertebral implant device of FIG. 29 in its fully expanded configuration 600. According to this exemplary embodiment, the expandable fusion device 600 is configured to have a first side and a second side that are independently expandable, such that a user may choose to expand (i.e., increase the height/distance between the first endplate 610 and the second endplate 620) only the first side of the implant or the second side of the implant. Expanding only the first side or second side of the implant will result in a so-called lordotic implant. In other words, the height of the expanded side is greater than the height of the unexpanded side, creating an angle (i.e., lordosis or a lordotic angle) between the first endplate 610 and the second endplate 620.

According to the exemplary embodiment shown in FIGS. 29-35, the expandable fusion intervertebral implant device 600 is dimensioned to span the width of a vertebral endplate, from the ipsilateral aspect of a vertebral body to the contralateral aspect of the vertebral body. By way of example, an expandable fusion intervertebral implant device 600 may range from about 40 mm to about 60 mm in length from leading end 602 to trailing end 604. The expandable fusion intervertebral implant device 600 is dimensioned to be placed in the intervertebral space from a lateral approach, e.g., direct lateral/transpsoas approach or anterolateral/anterior to psoas approach. By way of example, the width of the expandable fusion intervertebral implant device 600 may range from about 18 mm to about 28 mm from the first side to the second side of the expandable fusion intervertebral implant device 600. It is also contemplated that the expandable fusion intervertebral implant device 600 in its collapsed configuration has parallel or substantially parallel endplates, i.e., approximately 0° of lordosis. It is also contemplated that the expandable fusion intervertebral implant device 600 in its collapsed state has a lordotic angle between the endplates 610, 620. By way of example, the angle between the first endplate 610 and the second endplate 620 in a collapsed configuration may range from 0° to 15°.

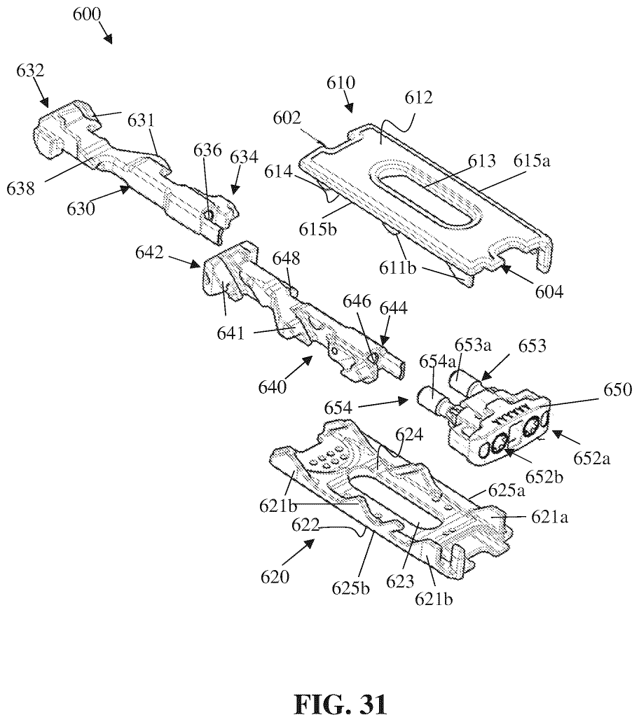

FIG. 31 is an exploded view of the expandable fusion intervertebral implant device 600 according to this embodiment. As best seen in FIG. 31, the expandable fusion intervertebral implant device 600 comprises a first endplate 610, a second endplate 620, a first expansion member 630, a second expansion member 640, a first actuator 653, a second actuator 654, and a housing 650.

The first endplate 610 has a top surface (or bone contacting surface) 612, an interior surface 614 opposite the top surface 612, a first side 615a, a second side 615b, a plurality of extensions 611a (FIG. 29) extending from the first side 615a along an edge of the endplate 610, and a plurality of extensions 611b (FIG. 30-31) extending from the second side 615b and offset from an edge of the endplate 610. The second endplate 620 has a bottom surface (or bone contacting surface) 622, an interior surface 624 opposite the bottom surface 622, a first side 625a, a second side 625b, a plurality of extensions 621a (FIG. 31) extending from the first side 625a offset from an edge of the endplate 620, and a plurality of extensions 611b (FIGS. 30-31) extending from the second side 625b along an edge of the endplate 620.

According to an exemplary embodiment, the first and second endplates 610, 620 are identical to each other in size and shape and are arranged opposite each other. According to an alternative embodiment, the first endplate 610 may have a height on the first side 615a that is greater than the height of the second side 615b of the first endplate 610 and/or greater than a maximum height of the second endplate 620. Each of the first and second endplates 610, 620 may further comprise openings 613, 623 (FIG. 31) extending from the top or bottom surface 612, 622 through the interior surface 614, 624 of the endplate 610, 620. According to one exemplary embodiment, each of the first and second endplates 610, 620 includes at least one central fusion aperture 613, 623, dimensioned and configured to allow bone growth through the first endplate 610, through a central region of the implant device 600 and through the second endplate 620. According to one embodiment best seen in FIG. 35, each of the first and second endplates 610, 620 comprises a porous bone contacting layer adjacent the top surface 612 and bottom surface 620, respectively.

The first expansion member 630 has a length comparable to the length of the first and second endplates 610, 620. The first expansion member 630 has a leading end 632, a trailing end 634, a plurality of curved surfaces 631 and an aperture 636 in the trailing end 634 that extends into at least a portion of the length of the first expansion member 630. The leading end 632 of the first expansion member 630 is beveled or tapered to assist with insertion of the expandable fusion intervertebral implant device 600 into an intervertebral space. The leading end 632 of the first extension member 630 extends beyond the leading ends 602 of the first and second endplates 610, 620 and forms the leading end of the overall expandable fusion intervertebral implant device 600. The leading end 632 of the first expansion member 630 may also comprise a dovetail or tongue and groove feature that mates with a corresponding feature on the first endplate 610. This dovetail or tongue and groove feature is configured to engage the corresponding feature on at least one of the first and second endplates 610, 620 to actively pull the first endplate 610 toward the second endplate 620 to “actively collapse” the expandable fusion intervertebral implant device 600.

According to an exemplary embodiment, the first expansion member 630 has a plurality of curved surfaces 631 that are spaced apart from each other and configured to engage the plurality of extensions 611a of the first side 615a of the first endplate 610 and the plurality of extensions 611a offset from the edge of the first side 625a of the second endplate 620. The first expansion member 630 may also comprise a cleat 638 (FIGS. 31-33) extending generally perpendicular to the longitudinal axis of the first expansion member 630 configured to mate with a corresponding cleat 648 (FIGS. 31-33) on the second expansion member 640 and allowing the first and second expansion members 630, 640 to be slidingly engaged with each other.

The second expansion member 640 includes features corresponding to the features described above with respect to first expansion member 630. That is, the second expansion member 640 has a leading end 642, a trailing end 644, a plurality of curved surfaces 641, an aperture 646 in the trailing end 644 that extends into at least a portion of the length of the second expansion member 640, and a cleat 648. The leading end 642 of the second expansion member 640 is beveled or tapered to assist with insertion of the expandable fusion intervertebral implant device 600 into an intervertebral space. The leading end 642 of the second extension member 640 extends beyond the leading ends 602 of the first and second endplates 610, 620 and forms the leading end of the overall expandable fusion intervertebral implant device 600. The leading end 642 of the second expansion member 640 may also comprise a dovetail or tongue and groove feature 633 (FIG. 35) that mates with a corresponding feature on the first endplate 610 and second endplate 620. This dovetail or tongue and groove feature 633 is configured to engage the corresponding feature on at least one of the first and second endplates 610, 620 to actively pull the first endplate 610 toward the second endplate 620 to “actively collapse” the expandable fusion intervertebral implant device 600.

Shapes, geometries and/or configurations of one or more wedges 631, 641 of expansion members 630, 640 and wedges 611, 621 of endplates 610, 620 may be customized to enhance expansion and collapsing. For example, as shown in FIG. 35, another active collapse feature 605 is shown. Specifically, the first and second endplates 610, 620 may include at least one extension 611c, 621c that is configured to interact with a wedge 641c of an expansion member 630, 640 during collapsing of the implant 600 such that the interaction of the extension 611c, 621c of the first and second endplate 610, 620 with the wedge 641c of the expansion member 630, 640 directs and assists in collapsing of the implant 600. It is to be understood that endplates 610, 620 and expansion members 630, 640 may include any number of active collapse mechanisms without departing from aspects of the disclosure.

The curved surfaces 641 of the second expansion member 640 are configured to engage the extensions 611b offset from the edge of the second side 615b of the first endplate 610 and the extensions 621b along the edge of the second side 621b of the second endplate 620. According to one exemplary embodiment, the first and second expansion members 630, 640 are identical in size and shape and may be arranged opposite each other such that the leading end 632, 642 and trailing end 634, 644 of the first and second expansion members 630, 640 are side by side, but the second expansion member 640 is flipped about the longitudinal axis of the expansion member. According to an exemplary embodiment, the apertures 636, 646 at the trailing ends 634, 644 of the first and second expansion members 630, 640 are dimensioned to receive a first portion 653a, 654a of the first and second actuator 653, 654, respectively. The apertures 636, 646 may be threaded to correspond to a thread form on the first portion 653a, 654a of the first and second actuators 653, 654.

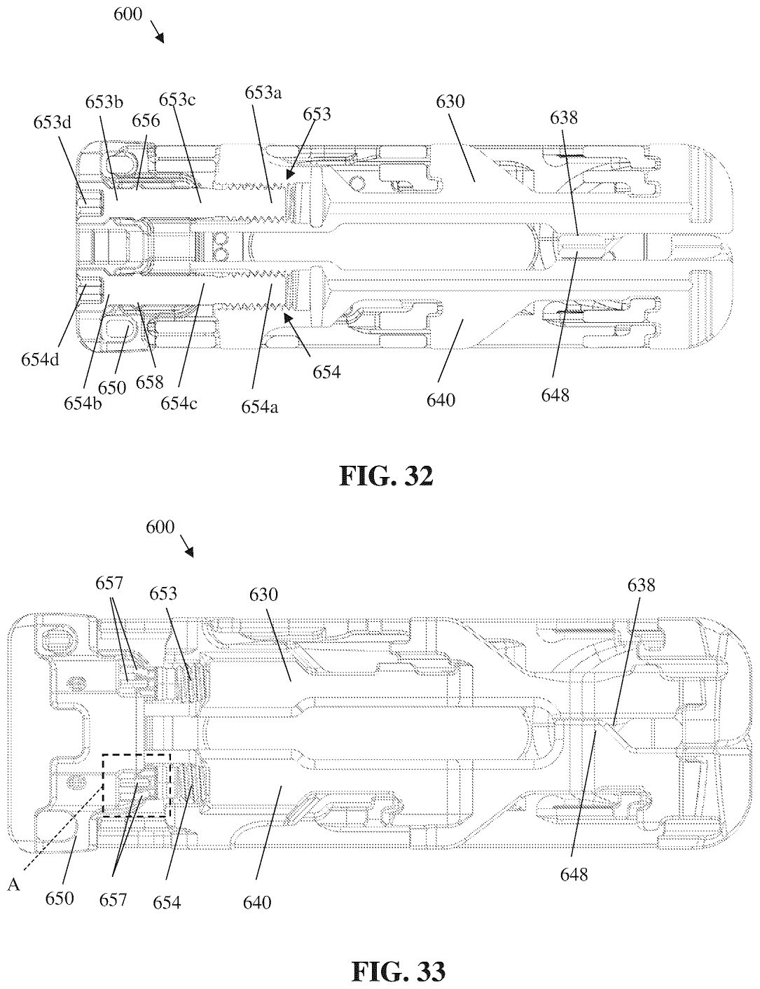

The first and second actuators 653, 654 each comprise a first portion 653a, 654a, a second portion 653b, 654b, and a middle region 653c, 654c (FIG. 32) between first portion 653a, 654a and the second portion (653b, 654b). According to an exemplary embodiment, the first and second actuators 653, 654 are identical to each other in size and shape. The first portions 653a, 654a of each of the first and second actuators 653, 654 are configured to engage the first and second expansion members 630, 640. For example, when the first actuator 653 is rotated in a first direction, the first expansion member 630 translates toward the housing 650 and when the first actuator 653 is rotated in a second direction, the first expansion member 630 translates away from the housing 650. Likewise, when the second actuator 654 is rotated in a first direction, the second expansion member 640 translates toward the housing 650 and when the second actuator 654 is rotated in a second direction, the second expansion member 640 translates away from the housing 650. The second portions 653b, 654b of each of the first and second actuators 653, 654 are substantially or completely captured within first and second apertures 652a, 652b (FIG. 31) in the housing 650, respectively. The trailing end of the second portion of each of the first and second actuators 653, 654 comprises an engagement feature 653d, 654d (FIG. 32), configured to receive the distal tip of an expansion driver that engages and rotates the first and second actuators 653, 654 in a first or a second direction. The engagement feature 653d, 654d at the trailing end of each of the first and second actuators 653, 654 is exposed through the corresponding aperture 652s, 652b in the housing 650 at the proximal face of the housing 650, which is also the trailing end face of the overall expandable fusion intervertebral implant device 600. Each of the actuators 653, 654 and corresponding expansion members 630, 640 may be completely independent of the other. The actuator 653, 654 and expansion member 630, 640 assemblies are configured to be actuated and translated independently, thereby increasing the height of only the first or the second side of the expandable fusion intervertebral implant device 600. Alternatively, the actuators 653, 654 may both be engaged and rotated simultaneously, causing parallel expansion of the expandable fusion intervertebral implant device 600. By way of example, the second portion 653b, 654b of each of the first and second actuators 653, 654 may include a lip or flange 656, 658 that has a greater circumference than the rest of the second portion 653b, 654b of the actuator 653, 654 that is retained in a corresponding pocket or channel within the first and second apertures 652a, 652b of the housing 650. The first and second actuators 653, 654 also each include a middle or central region 653c, 654c. The central region 653c, 654c may have a plurality of concave channels about the circumference of the middle region 653c, 654c of the actuator 653, 654.

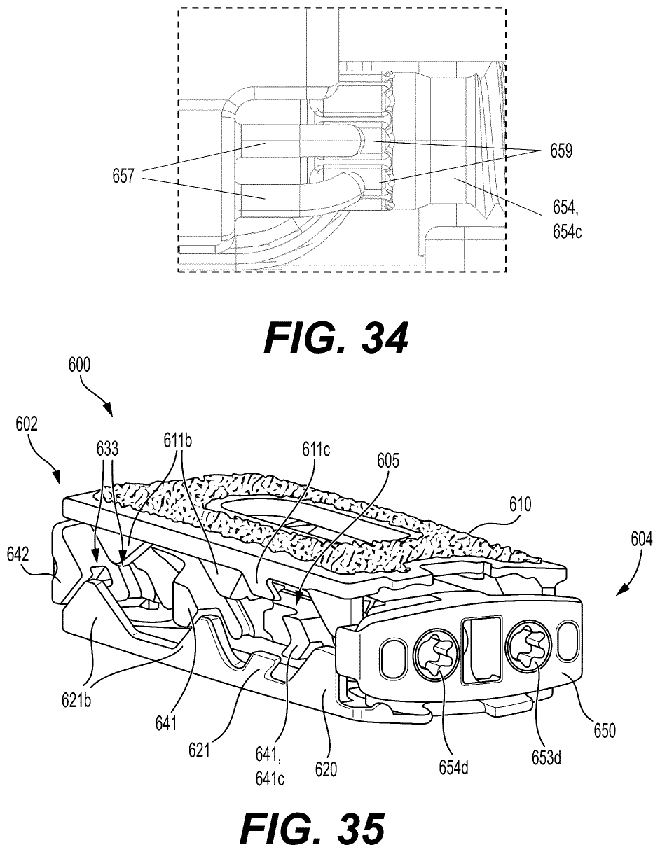

Housing 650 is configured to be positioned at trailing end 604 of expandable fusion intervertebral implant device 600. According to one embodiment, the housing 650 has a width from the first side to the second side of the expandable fusion intervertebral implant device 600 that is equal to the maximum width of the expandable fusion intervertebral implant device 600. The housing 650 includes a distal face and a proximal face, first and second apertures 652a, 652b (FIG. 31) that extend from the proximal face through the distal face of the housing 650, and a plurality of flexible extensions 657 (FIG. 33) extending from the distal face of housing 650 adjacent the first and second apertures 652a, 652b. The first and second apertures 652a, 652b are configured to retain second portions 653b, 654b of first and second actuators 653, 654, respectively. Each of the plurality of flexible extensions 657 extending from the distal face of housing 650 has a distal end configured to rest in one of the lobes or concave channels 659 (FIG. 34) about the circumference of the middle region 653c, 654c of actuator 653, 654. According to an exemplary embodiment, the plurality of flexible extensions 657 may have different lengths, such that flexible extensions 657 engage concave channels 659 in the middle region 653c, 654c of actuator 653, 654 at different locations. According to the exemplary embodiment, housing 650, including the plurality of flexible extensions 657, may be of unitary construction. The flexible extensions 657 are configured to restrict an undesired movement of first and second actuators 653, 654. The second portions 653b, 654b of first and second actuators 653, 654 are captive within housing 650 without requiring the use of a secondary lock or screw feature.

According to an exemplary embodiment, the entire expandable fusion intervertebral implant device 600 may be manufactured using additive manufacturing techniques. According to another exemplary embodiment, the expandable fusion implant may comprise a biocompatible metal. At least one of the endplates 610, 620 or expansion members 630, 640 may be treated to enhance the surface structure to facilitate bone ingrowth or ongrowth to the treated component. According to another exemplary embodiment, the entire expandable fusion intervertebral implant device 600 may be treated to enhance the surface structure to facilitate bone ingrowth or ongrowth to the expandable fusion intervertebral implant device 600.

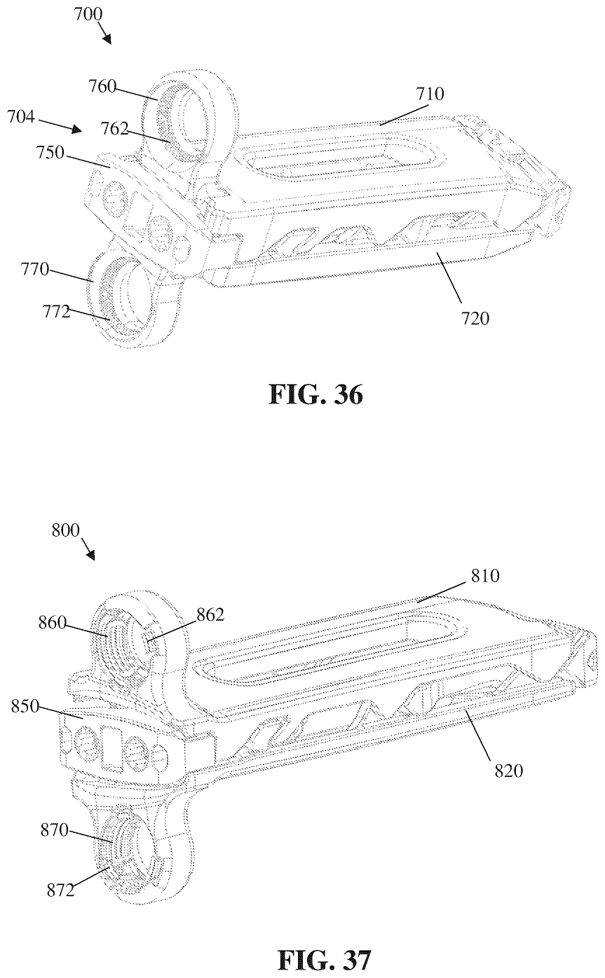

Turning now to FIG. 36, another embodiment of an expandable fusion intervertebral implant device 700 is shown. This embodiment includes features having substantial correspondence with the features of expandable fusion intervertebral implant device 600 (FIGS. 29-35), however, expandable fusion intervertebral implant device 700 includes a plated design. More specifically, expandable fusion intervertebral implant device 700 includes at least one fixation aperture 760 integrally formed with the first endplate 710 and at least fixation aperture 770 integrally formed with the second endplate 720. Each of the at least one fixation apertures 760, 770 is configured to receive a fixation element, e.g., a bone screw, blade, etc. therein for affixing the expandable fusion intervertebral implant device 700 to adjacent bone. The housing 750 may be positioned between the at least one fixation aperture 760 integrally formed with the first endplate 710 and the at least one fixation aperture 770 integrally formed with the second endplate 770. While shown as including two fixation apertures 760, 770, it is understood that each endplate 710, 720 may include any number of fixation apertures without departing from aspects of the disclosure. Additionally, it is also contemplated that only one of the first endplate 710 or second endplate 720 may include a fixation aperture.

In this embodiment, each of the at least one fixation apertures 760, 770 are shown including a canted coil mechanism 762, 772 as an anti-back out feature, but other types may be used to secure the expandable fusion intervertebral implant device 700 with respect to adjacent bone once it is placed and sized. Additional details of an exemplary canted coil locking mechanism can be found in International Patent Application No. PCT/US2009/040396, filed on Apr. 13, 2009, and published as WO 2009/126968 A1 on Oct. 15, 2009, which is hereby incorporated by reference as if set forth in its entirety herein. For example, some embodiments may include a polymeric member for ultrasonic welding. Additional details of an exemplary polymeric member can be found in International Patent Application No. PCT/US2018/040463, filed on Jun. 29, 2018, and published as WO 2019/006407 A1 on Jan. 3, 2019, which is hereby incorporated by reference as if set forth in its entirety herein.

Each of the at least one fixation apertures 760, 770 may be positioned about the endplates 710, 720 on a trailing end 704 of the expandable fusion intervertebral implant device 700. Further, each of the at least one fixation apertures 760, 770 may be centered about the endplates 710, 720 at the trailing end 704 or may be positioned off-centered. Additionally, each of the at least one fixation apertures 760, 770 may be configured to direct a bone screw positioned therein in a desired angulation and direction. In one example, the fixation apertures 760, 770 are shifted to an anterior portion of the expandable fusion intervertebral implant device 700 and the bone screw trajectory may be directed in a posterior direction, which allows the expandable fusion intervertebral implant device 700 to be used in an anterior to psoas (ATP) approach.

Turning now to FIG. 37, another embodiment of an expandable fusion intervertebral implant device 800 is shown. This embodiment includes features that substantially correspond to those described relative to expandable fusion intervertebral implant device 600 (FIGS. 29-35), however, expandable fusion intervertebral implant device 800 includes is plated in a manner similar to expandable fusion intervertebral implant device 700. More specifically, expandable fusion intervertebral implant device 800 includes at least one fixation aperture 860 integrally formed with the first endplate 810 and at least fixation aperture 870 integrally formed with the second endplate 820. Each of the at least one fixation apertures 860, 870 are configured to receive a fixation element, e.g., a bone screw, blade, etc., therein for affixing the expandable fusion intervertebral implant device 800 to adjacent bone. The housing 850 may be positioned between the at least fixation aperture 860 integrally formed with the first endplate 810 and the at least one fixation aperture 870 integrally formed with the second endplate 870.

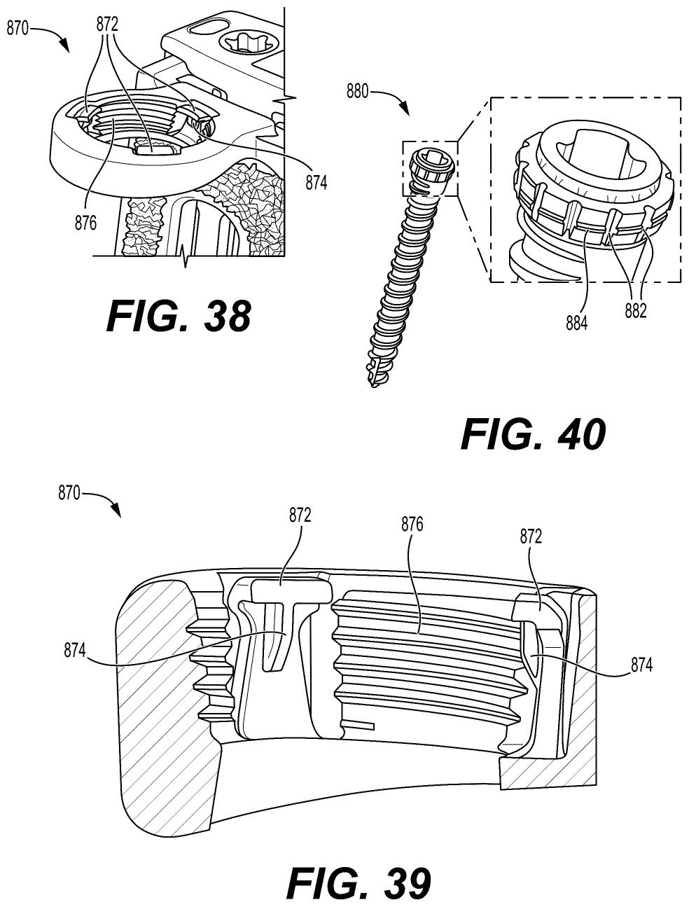

In this embodiment, each of the at least one fixation apertures 860, 870 are shown including at least one latch lock 862, 872 positioned therein. While the fixation apertures 860, 870 are shown including three latch locks 862, 872 per fixation aperture, it is to be understood that any number of latch locks 862, 872 may be included without departing from aspects of the disclosure. FIG. 38 shows an enlarged view of the fixation aperture 870 having latch locks 872 therein. FIG. 39 shows an enlarged cross-sectional view of the fixation aperture 870. As shown, each latch lock 872 includes a ratchet tab 874 for engaging scalloped cuts 882 (FIG. 40) on the head of a bone screw 880 (FIG. 40) thereby providing an anti-backout mechanism. The ratchet tabs 874 may project inwardly, toward the center of the fixation aperture 870 and/or distally (downwardly in the view as shown FIG. 39) toward the opposite end of the fixation aperture 870. The latch locks 872 may be configured to permit some degree of flexibility to allow the latch locks 872 to deflect or splay outwardly during bone screw 880 placement. Additionally, the fixation aperture 870 includes threads 876 for engaging a helical interface feature 884 on the head of the fixation element 880 to ensure bottoming out of the fixation element 880 within the fixation aperture 870. While the latch lock, ratchet tab, and threads 876 have been shown and described relative to the at least one fixation aperture 870, it is to be understood that the at least one fixation aperture 860 may include substantially the same features, and therefore description of which has been omitted for brevity. Latch locks 872, ratchet tabs 874, and threads 876 may be formed within the fixation aperture 870, and analogously within fixation aperture 860 via 3D printing.

In use, a fixation element 880 is inserted into a fixation aperture 860, 870 via a driver (not shown). As the fixation element 880 is inserted, the latch locks 872 deflect or splay outwardly and then snap back inwardly toward the center of the fixation aperture 860, 870 such that the rachet tab 874 of each latch lock 872 engages with a scalloped cut 882 of the fixation element 880, thereby providing rotational backout resistance. Additionally, the helical interface feature 884 of the fixation element 880 engages with threads 876 of the fixation aperture 860, 870, thereby providing a hard stop tactile feedback and backout resistance. Specifically, the helical interface feature 884 has a pitch that differs from the pitch of the threads 876 and causes cross-threading and/or binding to provide tactile feedback and ensure bottoming out of the fixation element 880 within the fixation aperture 860, 870.

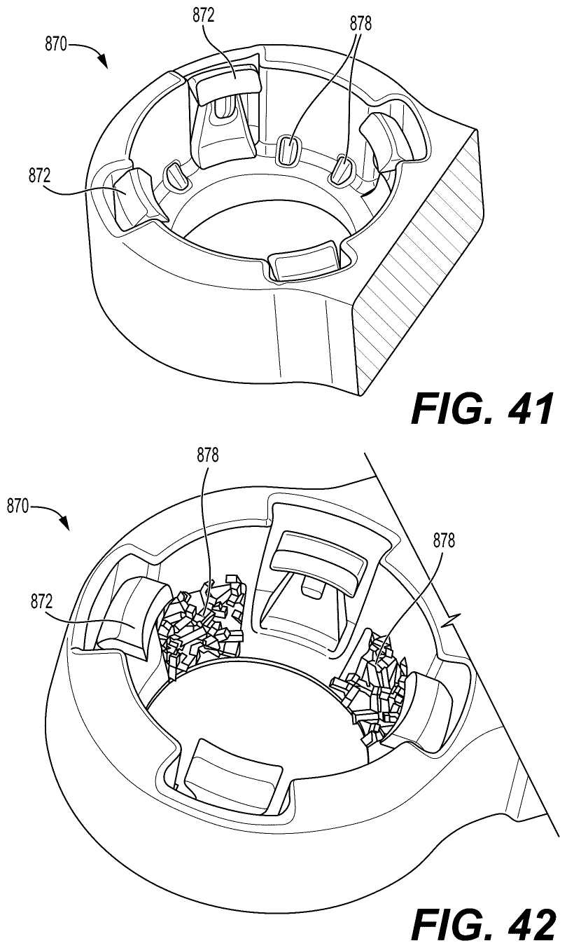

FIG. 41 shows another embodiment of anti-back out and hard stop features for the fixation apertures 860, 870. Specifically, this embodiment will again be discussed relative to fixation aperture 870 but is equally applicable to the fixation aperture 860. As shown, the fixation aperture 870 includes the latch locks 872 as in FIGS. 38-39, but instead of threads 876, includes ridges or bumps 878 may be formed proximate to the bottom of the fixation aperture 870 for mating with and/or engaging with cuts formed within the fixation element 880. The ridges or bumps 878 may be formed via 3D printing. The ridges or bumps 878 provide a hard stop in the form of a noticeable torque spike when the bottom of the head of the fixation element 880 engages with the ridges or bumps 878.

FIG. 42 shows another embodiment of the ridges or bumps 878. In this embodiment, the ridges or bumps 878 are in the form of micropores formed via 3D printing. The micropores provide a hard stop in the form of a noticeable torque spike when the bottom of the screw head engages with the micropores.

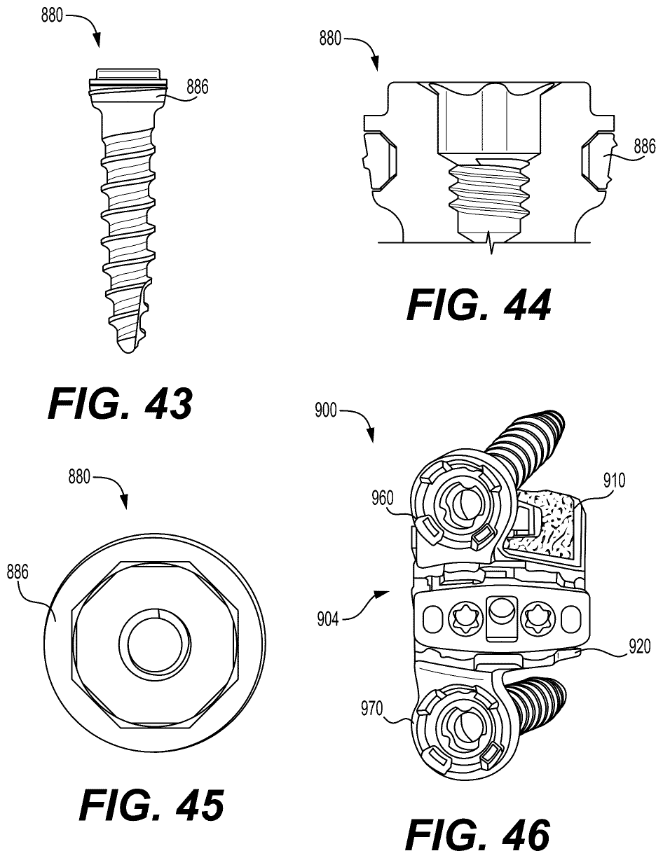

FIGS. 43-45 show another embodiment of an anti-backout mechanism in the form of a washer 886 configured to be formed substantially surrounding the head of the fixation element 880 and being received within a groove around the head. The washer 886 includes a helical interface feature 884. The helical interface feature of the washer 886 is configured to engage threads 876 (FIGS. 38-39) of the fixation aperture 870 similar to that described relative to FIG. 40. An inner surface of the washer 886 may be keyed and dimensioned such that sufficient counter torque to seat the fixation element 880 is provided by preventing rotation. This configuration allows the fixation element 880 to be driven into the fixation apertures of the expandable fusion intervertebral implant device 700, 800 and achieve a hard stop, but allows for toggle to account for post-operative settling of the bone screw 880. The fixation element 880 having washer 886 may be formed via 3D printing.

FIG. 46 shows an embodiment of an expandable fusion intervertebral implant device 900 including fixation apertures 960, 970 integrally formed with endplates 910, 920 wherein the fixation apertures 960, 970 are positioned on the trailing end 904 at the anterior side and configured to provide a screw trajectory toward the posterior side. Such an embodiment may be used in an anterior to psoas (ATP) approach.

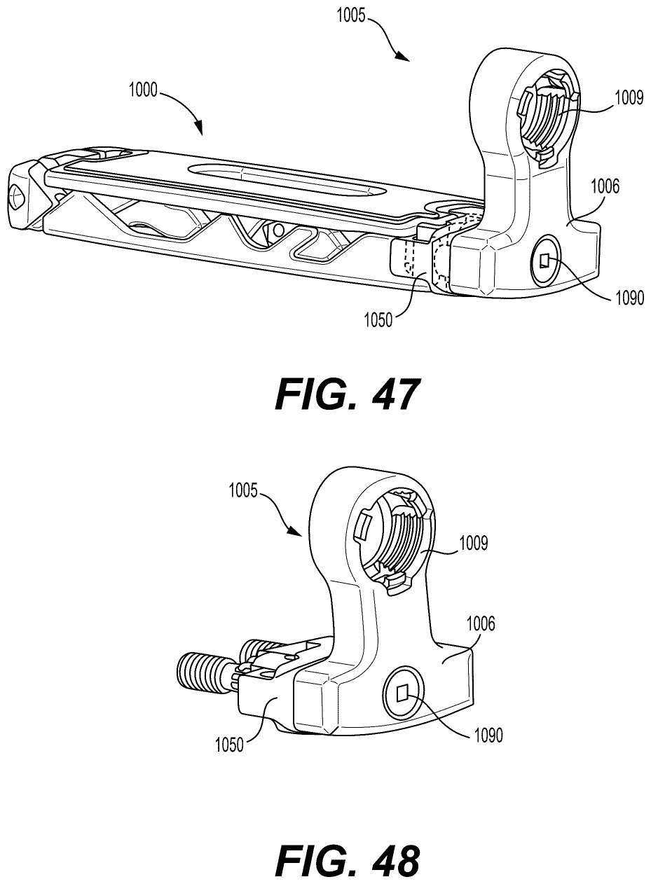

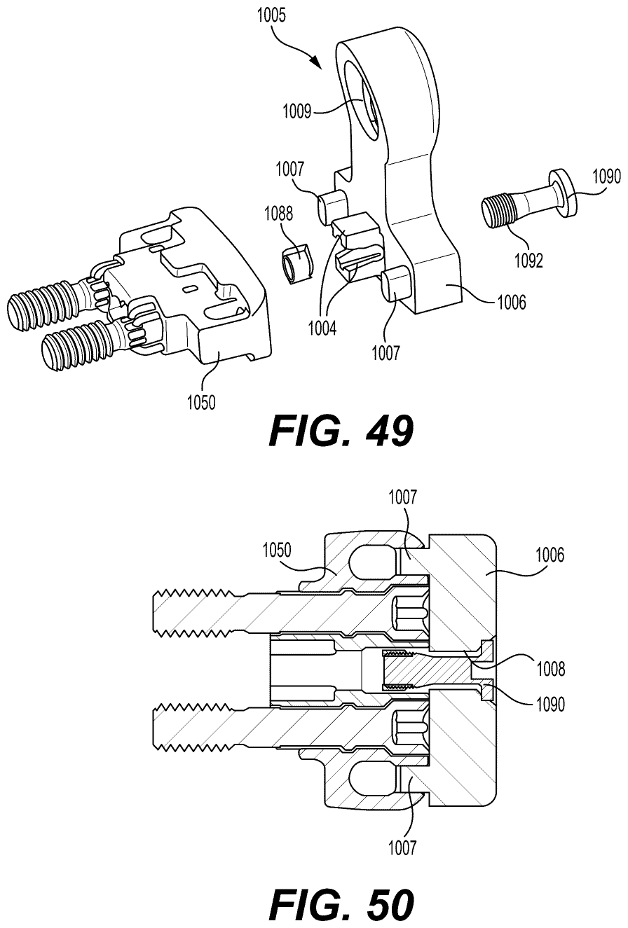

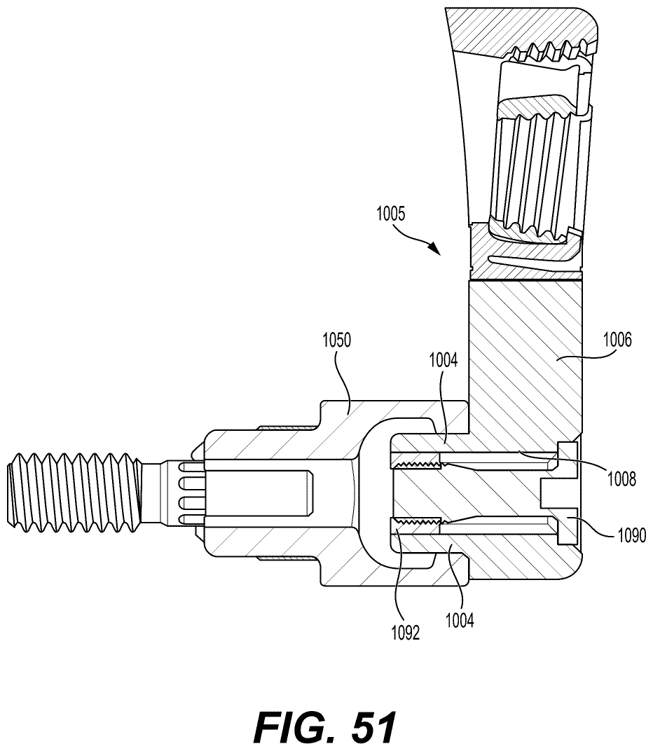

Turning now to FIGS. 47-51, an expandable fusion intervertebral implant device 1000 having a modular plate configuration is shown. Specifically, implant device 1000 is shown including a plate 1005 configured to be coupled to the housing 1050 of the expandable fusion intervertebral implant device 1000. The expandable fusion intervertebral implant device 1000 may be in the form of any of the embodiments discussed herein, e.g., shown in FIGS. 1-37. Plate 1005 includes a main body 1006 having at least one boss, tab, extension, and/or protrusion (hereinafter, “boss”) 1007 extending from a distal surface thereof (FIGS. 49-50). Boss 1007 is configured to be received within an aperture within the housing 1050. Main body 1006 also includes a pair of wings, tabs, extensions, or protrusions (hereinafter, “wings”) 1004 which also extend into an opening within the housing 1050. Main body 1006 may be substantially “T-shaped” or “t-shaped”, however, other shapes are also feasible and contemplated by the disclosure. The main body 1006 may also have an opening 1008 extending entirely through the main body 1006 and may be configured to be aligned with an aperture within the housing 1050.

Plate 1005 may also include at least one fixation aperture 1009 that is integrally formed with the main body 1006. Fixation aperture 1009 is configured to receive a fixation element, e.g., a bone screw, a blade, etc., therein to fix the plate 1005, and therefore, the expandable fusion intervertebral implant device 1000, to adjacent bone. The fixation aperture 1009 may be configured similarly to any of fixation apertures 760, 770, 860, 870, 960, 970 (FIGS. 36-46) such that fixation aperture 1009 may include any number of anti-backout mechanisms and bottom out feedback mechanisms, including a canted coil mechanism, a latch lock mechanism, threads, ridges, bumps, and/or micropores as described herein.

An attachment 1090 may be configured for insertion into opening 1008 (FIG. 50) of main body 1006 and into the opening within housing 1050 to fix plate 1005 to expandable fusion intervertebral implant device 1000. In some embodiments, attachment 1090 includes a threaded end 1092 configured to threadably engage with a complementary thread within the opening of housing 1050. In other embodiments, attachment 1090 is threaded within a nut 1092 positioned within and between wings 1004 of plate 1006. As attachment 1090 is threaded into opening 1008, nut 1092 is drawn away from housing 1050, thereby causing wings 1004 to separate and/or spread apart. As a result, an interference fit is created between wings 1004 and housing 1050.

The embodiment shown in FIGS. 47-51 is modular in that the plate 1005 is separate but couplable and removeable relative to the expandable fusion intervertebral implant device 1000. The plate 1005 is configured to be removeably coupled to housing 1050 after expandable fusion intervertebral implant device 1000 is expanded to a desired configuration. As a result, the method described herein may also include coupling the plate 1005 to the implant and fixing the plate 1005 to bone via a fixation element received within fixation aperture 1009. This modular design allows the surgeon the option of using a plated implant without the need to remove a non-plated implant and replacing it with a plated implant.

Although particular features and embodiments have been described in an effort to enable those with skill in the art to make and use the claimed invention, it should be understood that several variations, alterations or substitutions can be achieved to arrive at the present disclosure. Nothing in this description shall be construed as limiting the spirit and scope of the invention as set forth in the claims, below.

What is claimed is:

1. An expandable implant comprising:
a first endplate and a second endplate;
a first translating member moveably coupled to a first actuator;
a second translating member moveably coupled to a second actuator;
wherein the first translating member is configured to move independently from the second translating member; and
wherein the first translating member and the second translating member are configured to change a spatial relationship between the first endplate and the second endplate,
wherein the first translating member is configured to threadably receive a threaded portion of the actuator, the actuator further including a plurality of divots circumferentially disposed around a non-threaded portion of the actuator.

2. The expandable implant of claim 1, wherein at least one of the first endplate and the second endplate further comprise a porous bone engagement surface.

3. The expandable implant of claim 1, wherein at least one of the first translating member and the second translating member further comprise at least one wedge configured to interface with at least one of the first endplate and the second endplate.

4. The expandable implant of claim 1, wherein the first translating member is moveably coupled to the second translating member.

5. The expandable implant of claim 4, wherein the first translating member is moveably coupled to the second translating member by a dovetail coupling.

6. The expandable implant of claim 1, further comprising a housing configured to enclose at least a portion of the first actuator and at least a portion of the second actuator.

7. The expandable implant of claim 1, further comprising an anti-migration feature.

8. The expandable implant of claim 7, wherein the anti-migration feature comprises a plurality of flexible members configured to restrict the rotation of at least one of the first actuator and the second actuator.

9. The expandable implant of claim 8, wherein one or more of the first actuator and the second actuator further comprise lobes configured to receive at least a portion of the flexible member configured to restrict a movement thereof.

10. An expandable implant comprising:
a first endplate and a second endplate;
a first translating member disposed between the first endplate and the second endplate and moveably coupled to a first drive screw; and
a second translating member disposed between the first endplate and the second endplate and moveably coupled to a second drive screw,
wherein the first translating member is configured to move independently from the second translating member,
wherein the first translating member and the second translating member are configured to change a spatial relationship between the first endplate and the second endplate, and
wherein the first translating member is configured to threadably receive a threaded portion of the actuator, the actuator further including a plurality of divots circumferentially disposed around a non-threaded portion of the actuator.

11. The expandable implant of claim 10, wherein at least one of the first endplate and the second endplate further comprise a porous bone engagement surface.

12. The expandable implant of claim 10, wherein the first translating member further comprises at least one wedge.

13. The expandable implant of claim 10, wherein the second translating member further comprises at least one wedge.

14. The expandable implant of claim 10, wherein the first translating member is moveably coupled to the second translating member, and the first translating member and second translating member are configured to move independently of one another.

15. The expandable implant of claim 14, wherein the first translating member is moveably coupled to the second translating member by a dovetail.

16. The expandable implant of claim 10, further comprising a housing configured to enclose at least a portion of the first drive screw and at least a portion of the second drive screw.

17. The expandable implant of claim 10, further comprising an anti-migration feature.

18. The expandable implant of claim 17, wherein the anti-migration feature comprises a plurality of flexible members configured to restrict the rotation of at least one of the first drive screw and the second drive screw.

19. The expandable implant of claim 18, wherein one or more of the first drive screw and the second drive screw further comprise lobes configured to receive at least a portion of the flexible member configured to restrict a rotation thereof.

20. A method of treating a spinal deformity, comprising the steps:
accessing an intervertebral disc space via a lateral approach;
inserting an expandable implant into the intervertebral disc space,
the expandable implant having a first translating member moveably coupled to a first actuator, and a second translating member moveably coupled to a second actuator, with the first translating member and the second translating member configured to change a spatial relationship between the first endplate and the second endplate as they move; and
actuating at least one of the first actuator and the second actuator to change the spatial relationship between the first endplate and the second endplate, thereby changing a dimension of the expandable implant.

21. An expandable implant comprising:
a first endplate and a second endplate;
a first expansion member moveably coupled to a first actuator; and
a second expansion member moveably coupled to a second actuator,
wherein the first expansion member is configured to move independently from the second expansion member, and
wherein the first expansion member and the second expansion member are configured to change a spatial relationship between the first endplate and the second endplate.

22. The expandable implant of claim 21, further comprising:
a first fixation aperture integrally formed with the first endplate and configured to couple the first endplate to a first bone; and
a second fixation aperture integrally formed with the second endplate and configured to couple the second endplate to a second bone.

23. The expandable implant of claim 22, wherein the first fixation aperture and the second fixation aperture each include an anti-backout mechanism configured to prevent a respective fixation element disposed therein from backing out of the respective first fixation aperture and second fixation aperture.

24. The expandable implant of claim 21, further comprising:
a housing disposed between the first endplate and the second endplate.

25. The expandable implant of claim 24, further comprising:
a plate removably coupled to the housing, the plate including:
a main body; and
a fixation aperture.
